(19) United States
(12) Patent Application Publication
(10) Pub. No.: US 2010/0158808 A1
(43) Pub. Date: Jun. 24, 2010

(54) Altered Activity of Toll-Like Receptors

(76) Inventors: Jeffrey L. Platt, Rochester, MN (US); Geoffrey B. Johnson, Rochester, MN (US); Gregory J. Brunn, Rochester, MN (US)

Correspondence Address: FISH & RICHARDSON P.C., PO BOX 1022, MINNEAPOLIS, MN, 55440-1022, US

(21) Appl. No.: 12/633,392

(22) Filed: Dec. 8, 2009

Related U.S. Application Data
(63) Continuation of application No. 10/559,423, filed on Feb. 1, 2007, now abandoned.
(60) Provisional application No. 60/478,067, filed on Jun. 12, 2003.

(51) Int. Cl. G01N 33/567; C12Q 1/68; A61K 49/00

(52) U.S. Cl. 424/9.2; 435/7.21; 435/6

(57) ABSTRACT

Materials and Methods for modulating TLR activity are described, as well as methods for reducing body fat and increasing bone density.

### CROSS-REFERENCE TO RELATED APPLICATIONS

This application is a continuation of U.S. application Ser. No. 10/559,423, filed Feb. 1, 2007, which is a National Stage application under 35 U.S.C. §371 and claims benefit under 35 U.S.C. §119(a) of International Application No. PCT/US2004/018859 having an International Filing Date of Jun. 10, 2004, which claims the benefit of priority of U.S. Provisional Application Ser. No. 60/478,067 having a filing date of Jun. 12, 2003.

### STATEMENT AS TO FEDERALLY SPONSORED RESEARCH

Funding for the work described herein was provided in part by the National Institutes of Health, grant numbers HL46810 and AI53733. The federal government has have certain rights in the invention.

This invention was made with government support under grant nos. HL46810 and AI53733, awarded by the National Institutes of Health. The government has certain rights in the invention.

### TECHNICAL FIELD

This invention relates to methods for altering the activity of toll-like receptors (TLR), and more particularly to methods for reducing the activity of TLR4.

### BACKGROUND

The Toll family of proteins is remarkably conserved across the taxonomic kingdoms. This family includes the invertebrate Toll proteins, the vertebrate Toll-like receptors, and the plant resistance genes (Hoffmann and Reichhart (2002) Nat. Immunol. 3:121-126; Akira et al. (2001) Nat. Immunol. 2:675-680; and Hulbert et al. (2001) Annu. Rev. Phytopathol. 39:285-312). Many of these proteins have homologous domains and signaling pathways, which are used to trigger inflammatory and immunological responses. However, the function of these proteins extends beyond host defense.

### SUMMARY

The invention is based on the discovery that complex saccharides synthesized as components of the normal extracellular matrix (ECM) can inhibit the ability of soluble heparan sulfate (HS) to stimulate cellular signaling via TLR4. These complex saccharides may be proteoglycans that represent a large component of the ECM, including, without limitation, HS- and chondroitin sulfate-protoglycans and hyaluronic acid. In addition, inhibition of TLR4 leads to increased bone density and decreased fat mass. Thus, inhibitors of TLR4 can be used to treat osteoporosis or obesity.

1. In one aspect, the invention features a method of identifying a compound that increases bone density. The method includes (a) contacting a cell with a test compound and monitoring the activity of a TLR (e.g., TLR2, TLR4, or TLR9) in the cell in response to an agonist, (b) administering the compound to a non-human subject (e.g., a rodent) if activity of the TLR in the cell is reduced relative to the level of activity of the TLR in the absence of the compound, and (c) identifying the compound as useful for increasing bone density if bone density in the non-human subject is increased relative to bone density in a corresponding subject to which the candidate compound was not administered. Monitoring activity of TLR4 is particularly useful. Monitoring TLR activity can include measuring expression of a cytokine or a chemokine. The test compound can be a glycosaminoglycan, a glycoprotein, a polysaccharide, a polypeptide, or a nucleic acid. The glycoprotein can include hyaluronic acid (e.g., a hyaluronic acid-protein conjugate), HS (e.g., a HS-protein conjugate), or chondroitin sulfate. The polypeptide can be an anti-CD14 antibody. The nucleic acid can be polymerized. The test compound can be a protease inhibitor (e.g., an elastase inhibitor). The test compound can be a heparanase, or the test compound can modulate the sulfation of HS. The test compound can be a modified lipid A molecule.

The invention also features a method of identifying a compound that decreases fat mass. The method includes (a) contacting a cell with a test compound and monitoring the activity of a TLR in the cell in response to an agonist, (b) administering the compound to a non-human subject if activity of the TLR in the cell is reduced relative to the level of activity of the TLR in the absence of the compound, and (c) identifying the compound as useful for decreasing fat mass if the fat mass in the non-human subject is decreased relative to the fat mass in a corresponding subject to which the test compound was not administered.

In another aspect, the invention features a method of identifying a compound for treatment of osteoporosis or obesity. The method includes (a) administering a test compound to a non-human subject, (b) monitoring activity of a TLR in response to an agonist in the non-human subject, and (c) identifying the test compound as useful for treatment of osteoporosis or obesity if activity of the TLR is decreased in the non-human subject relative to that of a corresponding non-human subject to which the test compound was not administered.

A method for reducing the activity of TLR4 also is featured. The method includes contacting a cell with an amount of a composition effective to reduce TLR4 activity, wherein the composition includes an ECM preparation. The ECM preparation can include intact HS. The method further can include monitoring TLR4 activity in the cell by, for example, measuring the expression of a cytokine (e.g., an interleukin or TNF-α) or a chemokine (e.g., IP10).

In yet another aspect, the invention features a method for reducing body fat in a subject (e.g., a human). The method includes administering to the subject an amount of a composition effective to inhibit TLR4 activity in the subject. The composition can include one or more components of an ECM. For example, the composition can include a HS-protein conjugate or a hyaluronic acid-protein conjugate. The composition can include an anti-CD14 antibody. The composition can include a protease inhibitor (e.g., an elastase inhibitor). The composition can include a heparanase, a compound that modulates the sulfation of HS, or a lipid A analogue. The method further can include monitoring body fat in the subject.

The invention also features a method for increasing percent lean body mass in a subject (e.g., a human). The method includes administering to the subject an amount of a composition effective to inhibit TLR4 activity in the subject. The composition can include one or more components of an ECM. For example, the composition can include a HS-protein conjugate or a hyaluronic acid-protein conjugate. The composition also can include an anti-CD14 antibody. The composition can include a protease inhibitor (e.g., an elastase inhibitor). The composition can include a heparanase, a compound that modulates the sulfation of HS, or a lipid A analogue. The method further can include monitoring percent lean body mass in the subject.

In yet another aspect, the invention features a method for increasing bone density or reducing bone loss in a subject (e.g., a human). The method includes administering to the subject an amount of a composition effective to inhibit TLR4 activity in the subject. The composition can include one or more components of an ECM (e.g., a HS-protein conjugate or a hyaluronic acid-protein conjugate). The composition also can include an anti-CD14 antibody. The composition can include a protease inhibitor (e.g., an elastase inhibitor). The composition can include a heparanase, a compound that modulates the sulfation of HS, or a lipid A analogue. The method further can include monitoring bone density in the subject.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing the level of reporter activity in HEK 293 cells that were stably transfected with TLR4/MD2 and/or CD14 as indicated, transiently transfected with nuclear factor-kappaB- (NFκB-) luciferase and internal control Renilla-luciferase reporter plasmids, and tested for response to PBS, HS, LPS, or recombinant human IL-1α.

FIG. 2A is a graph plotting the level of reporter activity in HEK/TLR4(+) cells transfected with NFκB- and control-luciferase reporter plasmids and then stimulated with intact HS or LPS, or with HS or LPS that had been treated with nitrous acid (HNO₂) at pH 4.0 or pH 1.5. Controls were treated with PBS vehicle. FIG. 2B is a graph showing the level of reporter activity in HEK/TLR4(+) cells that were transfected with NFκB- and control-luciferase reporters and then treated with intact HS, HS digested with heparanase, inactive (boiled) enzyme, or PBS. FIG. 2C is a graph showing the effect of PBS, HS, depolymerized HS (nitrous acid, pH 1.5), and recombinant human IL-1α on cell surface expression of TLR4/MD2 in HEK/TLR4(+) cells. Expression of TLR4 on the cell surface was determined by flow cytometry using a monoclonal antibody specific to the TLR4/MD2 complex. Values are the mean of triplicate wells and are representative of three separate experiments.

FIG. 3A is a graph showing the level of reporter activity in HEK/TLR4(+) cells that were cultured on ECM or fibronectin (FN) and treated with increasing amounts of HS. Control HEK 293 cells not expressing TLR4 and MD2 (No TLR4) also were used. FIG. 3B is a graph showing the level of reporter activity in HEK/TLR4(+) cells that were cultured on ECM or FN and treated with increasing amounts of LPS. Control HEK 293 cells not expressing TLR4 and MD2 (No TLR4) also were used. FIG. 3C is a graph showing the level of p38 MAP kinase activity in RAW 294 cells that were cultured with ECM or FN and then stimulated with HS for the indicated times. The amounts of activated (phosphorylated) and total p38 MAPK were determined by immunoblotting with anti-phospho-p38 antibodies and anti-p38 antibodies, followed by densitometry scanning of immunoblot films. Data shown are representative of 3 separate experiments.

FIG. 4 is a graph showing levels of reporter activity in HEK 293 cells that were cultured in wells coated with ECM, transfected with NFκB- and control-luciferase reporter plasmids, and treated with IL-1α or TNFα. Data are expressed as percentage of the activity obtained in cells grown on control fibronectin-coated plates. Results from HEK/TLR4(+) cells stimulated with HS or LPS are shown for comparison.

FIG. 5A is a graph plotting NFκB-luciferase reporter plasmid activity in TLR4-transfected HEK 293 cells in response to plating on ECM, pre-treating with elastase, and treating with HS. FIG. 5B is a graph plotting reporter plasmid activity in TLR4-transfected HEK 293 cells in response to plating on ECM, pre-treating with elastase, and treating with LPS.

FIG. 6 is a graph showing levels of reporter activity in HEK/TLR4(+) cells that were cultured in wells coated with ECM or fibronectin (FN) and transfected with NFκB- and control-luciferase reporter plasmids. The cells were treated with the indicated concentrations of elastase, with or without HS. Data are the means from triplicate wells and are representative of 3 experiments. *p<0.05 compared to no elastase on ECM. # p<0.05 compared to low dose elastase on ECM.

FIG. 7 is a graph plotting reporter plasmid activity in transfected HEK 293 cells in response to plating on various extracellular matrices and treating with HS.

FIG. 8 is a graph showing the level of reporter activity in HEK/TLR4(+) cells that were transfected with NFκB- and control-luciferase genes and then stimulated with ECM fragments generated by treatment with PBS (C), elastase (E), heat inactivated elastase (ΔE), or elastase-generated ECM fragments incubated with recombinant heparanase (E→H). *p<0.05 compared to control; #p<0.05 compared to (E→H).

FIG. 9 is a graph showing levels of CD40 expression on the surface of dendritic cells treated with PBS, LPS, unmodified HS, HS modified by N-desulfation followed by N-acetylation (NDSNAc), HS modified by complete desulfation followed by replacement of N-sulfation (CDSNS), HS modified by complete desulfation followed by N-acetylation (CDSNAc), or each modified HS combined with unmodified HS.

FIG. 10 is a graph showing the level of systemic inflammatory response induced by HS via TLR4 in wild type (TLR4+) or TLR4 nonsignaling mutant (TLR4−) mice that were injected with D-galactosamine plus TLR4 agonists (HS or LPS), or the TLR9 agonist CpG DNA. Control injections included D-galactosamine plus the PBS vehicle, chondroitin sulfate (CS), heparin (Hep), or LALF, a protein that specifically binds to and neutralizes LPS but not HS. Each group included four or five animals. When no deaths occurred, no bar is indicated on the graph. Results are representative of two experiments. *p<0.05 using Fisher's exact test when compared with TLR4-negative controls.

FIG. 11 is a graph showing the level of TNF-α released by immature dendritic cells that were treated with elastase. Cells were obtained from wild type (TLR4⁺) or TLR4 nonsignaling mutant (TLR4⁻) female mice, and were cultured on confluent porcine aortic endothelial cells (PAEC). The cocultures were treated with HS, LPS, the TLR9 agonist CpG DNA (CpG), the TLR2 agonist zymosan (Zym), chondroitin sulfate (CS), heparin (Hep), or the ECM-mobilizing enzymes heparanase (H'ase) or elastase (El). All conditions were tested in triplicate wells and the mean concentrations of TNF-α from the supernatant, as measured by ELISA, are shown. TNF-α concentrations below assay detection limits were set at 0 and no bar is indicated on the graph. Data are representative of two experiments.

FIG. 12 is a graph showing serum TNF-α concentrations in wild-type (TLR4⁺) or TLR4-deficient (TLR4⁻) mice that were injected with elastase (El), HS, LPS, CpG DNA, heparin (Hep), or the PBS vehicle only. Mean serum TNF-α concentrations 1 hour after treatment are shown. TNF-α concentrations were measured in triplicate and the results are representative of two experiments. Serum TNF-α levels below the sensitivity of the assay were assigned a value of 0, and no bar is indicated on the graph.

FIG. 13A is a graph showing total body, thoracic, abdominal, and pelvic fat mass in wild type (TLR4+) and TLR4 mutant (TLR4−) mice. *P<0.05. FIG. 13B is a graph showing percent body fat in thoracic regions, abdominal regions, pelvic regions, and total bodies of wild type and TLR4 mutant mice. *P<0.05; **P≦0.01. FIG. 13C is a graph showing total body, thoracic, abdominal, and pelvic lean mass in wild type and TLR4 mutant mice. FIG. 13D is a graph showing total mass in thoracic regions, abdominal regions, pelvic regions, and total bodies of wild type and TLR4 mutant mice.

### DETAILED DESCRIPTION

In general, the invention provides methods and materials for altering TLR (e.g., TLR 2, 4, or 9) activity. TLR4 is the main receptor on cells that transduces signals delivered by endotoxin (lipopolysaccharide (LPS)) and other bacterial products. TLR4 is expressed on adipocytes and osteoblasts and their common precursor, the stromal cell. TLR4 also is expressed on macrophages, dendritic cells, and osteoclasts and their common precursor in the bone marrow. TLR4 does not appear to have high affinity for LPS, suggesting other molecules may facilitate interaction of LPS with TLR4. One such molecule may be MD-2, a soluble protein that is non-covalently associated with TLR4 on the surface of cells and is required for TLR4 recognition of LPS.

As described herein, TLR4 plays a role in normal homeostasis and/or regulation of bone density or body fat in the absence of infection. Inhibition of TLR4 or targets in the TLR4 signaling pathway (e.g., CD14) can reduce body fat in an animal and improve (i.e., increase) bone density when given long term. Consequently, TLR4 inhibitors can be used to treat obesity, osteoporosis, and related conditions.

Obesity is a poorly understood condition that is associated with sedentary life styles and high caloric intake. There is an increasingly severe epidemic of obesity in the United States, with three out of five American adults being overweight and one out of three being obese. In 1995 it was estimated that over $50 billion was spent on obesity related health care in the U.S., amounting to approximately 6% of entire national health care expenditure. Obesity is a causative factor for other serious conditions including, for example, atherosclerosis/cardiovascular disease, gastroesophageal reflux disease, diabetes type II, apnea (obstructive sleep apnea), asthma, gallbladder disease, non-alcoholic steatohepatitis, infertility/polycystic ovarian syndrome, certain cancers (e.g., breast cancer, colon cancer, endometrial cancer, kidney cancer, and esophageal cancer), pseudotumor cerebri, deep vein thrombosis, panniculitis/cellulitis, dyslipidemia, stress incontinence, and adverse psychosocial effects. Metabolic syndrome also is related to obesity, and is believed to affect about 20-25% of adults in the U.S. This syndrome is characterized by observation of a group of metabolic risk factors in one person. These risk factors include central obesity (excessive fat tissue in and around the abdomen), atherogenic dyslipidemia (blood fat disorders—mainly high triglycerides and low HDL cholesterol—that foster plaque buildups in artery walls), increased blood pressure (130/85 mmHg or higher), insulin resistance or glucose intolerance, prothrombotic state (e.g., high fibrinogen or plasminogen activator inhibitor [-1] in the blood), and proinflammatory state (e.g., elevated high-sensitivity C-reactive protein in the blood). Reducing obesity by inhibiting TLR4 can result in improved health, enhanced quality of life, and increased life expectancy in subjects having the above obesity-related conditions. For example, the experiments described in Example 16 show that mice lacking functional TLR4 displayed reduced abdominal adiposity. Thus, the methods provided herein may be useful to reduce the high central obesity that is observed with metabolic syndrome. Furthermore, TLR4 inhibitors can be used to treat patients taking chronic steroids or other drugs that increase adiposity.

In addition to the conditions described above, osteoporosis and/or low bone mass are a threat for over 55% of the U.S. population aged 50 and older. Direct medical costs for treating fractures resulting from osteoporosis are $17 billion annually. Other conditions that can put a subject at risk for bone fracture include osteogenesis imperfecta, radiation treatment (e.g., radiation treatment of cancer patients), organ transplantation (e.g., bone marrow transplant), renal disease, and hypercalciuria. As described herein, TLR4 mutant mice displayed increased bone density relative to wild type mice. Thus, treatment with a TLR4 inhibitor may be useful to increase bone density and reduce or prevent the incidence of fractures in subjects having such conditions.

Methods for Identifying Compounds that Inhibit TLR Activity

Compounds that inhibit TLR activity can be identified using in vitro or in vivo methods, or combinations of in vitro and in vivo methods. For example, a compound that increases bone density or decreases fat mass can be identified by contacting a cell in vitro with a test compound in the presence of an agonist (e.g., lipid A or a mono or disaccharide such as those disclosed in U.S. Patent Publication 20020077304), and then monitoring the activity of the TLR. Cells that can be used in the methods of the invention include cell lines such as human embryonic kidney cells (e.g., HEK293 cells), adipocyte cell lines, macrophage cell lines (e.g., RAW), or primary cell cultures. In some embodiments, cells can be obtained from a particular subject to be tested. Compounds shown to inhibit TLR activity can be administered to a non-human subject for in vivo studies. Alternatively, test compounds can be directly administered to a non-human subject.

Compounds may inhibit TLR directly or indirectly (e.g., by inhibiting an upstream molecule). Test compounds can include, for example, small molecules, an ECM preparation, glycosaminoglycans, glycoproteins, polysaccharides, polypeptides, and nucleic acids (e.g., polymerized nucleic acids). For example, a glycoprotein can include hyaluronic acid or a hyaluronic acid-protein conjugate, HS or a HS protein conjugate, or chondroitin sulfate. HS and other glycosaminoglycans can be commercially obtained, purified from a biological sample, or prepared synthetically. See, for example, Yates et al. [(2004) J. Med. Chem. 47:277-280], which describes “chemicoenzymatic” preparation of structurally diverse heparan sulfate analogue libraries from heparin.

Polymerized molecules may be useful due to their larger size. Intact HS, i.e., repeating glucosamine and hexuronic acid units linked to a core protein in the ECM, can be particularly useful. As described herein, HS acts more specifically than heparin, a related glycosaminoglycan. Without being bound to a particular mechanism, the data shown herein indicate that intact and anchored proteoglycans, such as exist in normal ECM, are inhibitors of TLR signaling. This contrasts with the soluble forms of such proteoglycans, which act as agonists of TLR signaling. Inflammation induces conditions that are conducive to mobilization of ECM proteoglycans, including, for example, oxidative stress, tissue damage, localized low pH, and release of matrix degrading enzymes by tissues and cells of the immune system. These inflammatory conditions are permissive for TLR activation by solubilized complex saccharides and proteoglycans.

Polypeptides that inhibit TLRs can include anti-CD14 polypeptides and antibodies (e.g., IC14, WT14, or ab8103). See, for example, U.S. Pat. No. 5,869,055, WO 02/42333, and WO 01/72993. CD14 aids in the interaction of LPS with cells. CD14 was originally reported to be the LPS receptor since it binds LPS with high affinity. However, because CD14 is a glycosylphosphatidylinositol (gpi)-anchored protein and lacks an intracellular signaling domain, it cannot transduce a signal by itself. Both the anchored form and a soluble form of CD14 can aid TLR4 recognition of LPS.

Analogues of agonists such as lipid A, fibronectin EDA, fibrinogen, or taxol also can be used to inhibit TLR. For example, the lipid A analogues alpha-D-glucopyranose, 3-O-decyl-2-deoxy-6-O-[2-deoxy-3-O-[(3R)-3-methoxydecyl]-6-O-methyl-2-[[(11Z)-1-oxo-11-octadecenyl]amino]-4-O-phosphono-beta-D-glucopyranosyl]-2-[(1,3-dioxotetradecyl)amino]-1-(dihydrogen phosphate) tetrasodium salt (E5564) and 6-O-[2-deoxy-6-O-methyl-4-O-phosphono-3-O—[(R)-3-Z-dodec-5-endoyloxydecl]-2-[3-oxo-tetradecanoylamino]-beta-O-phosphono-alpha-D-glucopyranose tetrasodium salt (E5531) can be used to inhibit TLR4. See, Mullarkey et al., J. Pharmacol. Exp. Ther. 2003, 304(3):1093-102. In addition, the lipid A analogues SDZ880.431 (3-aza-lipid X-4-phosphate), E5564, E5531, and RsDPLA (diphosphoryl lipid A derived from non-toxic LPS of Rhodobacter sphaeroides) can be used as test compounds to inhibit TLR4. See, e.g., Manthey et al. (1993) Infect. Immun. 61:3518-3526; Perera et al. (1993) Infect. Immun. 61:2015-2023; Liang et al. (2003) J. Clin. Pharmacol. 43:1361-1369; Wasan et al. (2003) Antimicrob. Agents Chemother. 47:2796-2803; Uehori et al. (2003) Infect. Immun. 71:4238-4249; Wong et al. (2003) J. Clin. Pharmacol. 43:735-742; Qureshi et al. (1991) Infect. Immun. 59:441-444; and Johnson et al. (2002) J. Immunol. 168:5233-5239. In other embodiments, the test compound can be an antibiotic (e.g., geladamycin). See, Vega and Maio, Mol. Biol. Cell, 2003, 14:764-773.

As described herein, ECM can inhibit TLR4 activity, while solubilized ECM components (e.g., solubilized HS) can activate TLR4. Proteases that cleave the ECM can release ECM components that may lead to activation of TLR4 signaling. Elastase (e.g., neutrophil or pancreatic elastase) is an example of such a protease. Thus, inhibitors of proteases such as elastase can be used to reduce the level of TLR4 activity in a subject. Inhibitors of elastase include, for example, elastase inhibitor I [Boc-Ala-Ala-Ala-NHO-Bz; see, Schmidt et al. (1991) In Peptides (Giralt and Andreu, eds.) 100:761], elastase inhibitor II [MeOSuc-Ala-Ala-Pro-Ala-CMK; see, Navia et al. (1989) Proc. Natl. Acad. Sci. USA 86:7; and Williams et al. (1987) J. Biol. Chem. 262:17178], elastase inhibitor III [MeOSuc-Ala-Ala-Pro-Val-CMK; see, Fletcher et al. (1990) Am. Rev. Respir. Dis. 141:672; Stein and Trainor (1986) Biochemistry 25:5414; and Powers et al. (1977) Biochim. Biophys. Acta 485:15], ONO-5046 [Suzuki et al. (1998) Kidney International 53:1201-1208], Epil-9, diisopropyl-phosphofluoridate, and alkyl isocyanates. Elastase inhibitors also can be identified using, for example, the method disclosed by Roberts et al. [(1992) Proc. Natl. Acad. Sci. USA 89:2429-2433].

Other enzymes that can degrade ECM and release soluble components such as HS include, for example, matrix metalloproteases [e.g., MMP1 (a.k.a. interstitial collagenase or fibroblast collagenase), MMP2 (a.k.a. 72 kD, collagenase type 4, collagenase type 4A, 72 kD gelatinase, gelatinase A, neutrophil gelatinase, CLG4, CLG4A, and TBE-1), MMP3 (a.k.a. stromelysin-1, transin-1, SL-1, PTR1 protein, gelatinase, and proteoglycanase), MMP7 (a.k.a. matrilysin, pump-1 protease, uterine metalloproteinase, and matrin), MMP9 (a.k.a. gel B, 92 kD gelatinase, collagenase 92 kD type IV, 92 kDa gelatinase, 92 kDa type IV collagenase, gelatinase B, macrophage gelatinase, and type V collagenase), and MMP13 (a.k.a. collagenase 3)]. See, e.g., De Ceuninck et al. (2003) Arthritis Rheum. 48:2197-2206. Additional examples of EMC-degrading enzymes include, without limitation, serine proteases such as plasmin, glycosidases, lyases (e.g., K5 lyase), endo-beta-d-glucuronidase (heparanase), heparitinase I, heparitinase II, and heparitinase III. See, e.g., Murphy et al. (2004) J. Biol. Chem. (published online ahead of print); Nardella et al. (2004) Biochemistry 43:1862-1873; Whitelock et al. (1996) J. Biol. Chem. 271:10079-10086; Li et al. (2002) Cell 111:635-646; and Endo et al. (2003) J. Biol. Chem. 278:40764-40770. Other matrix degrading enzymes can be identified using assays that are commercially available from, for example, BIOalternatives (Gencay, France).

Since the above molecules can degrade ECM and solubilize ECM components, potentially activating TLR4, inhibitors of these enzymes can be used to reduce TLR4 activity. For example, matrix metalloproteases can be inhibited by marimastat (Wojtowicz-Praga et al. (1997) Invest. New Drugs 15:61-75). Inhibitors of heparanase include, for example, antibody 733, suramin, and RK-682 (from RK99-A234). See, e.g., Zetser et al. (2004) J. Cell Sci. 117(Pt 11):2249-2258; Ishida et al. (2004) J. Antibiot. (Tokyo) 57:136-142; and Nardella et al. (supra). In some embodiments, however, heparanase can be used to degrade soluble HS, and thus heparanase itself can be useful as an inhibitor of TLR4. Without being bound by a particular mechanism, the use of heparanase as an inhibitor of TLR4 may depend on the conditions under which heparanase can act to completely degrade HS to molecules too small to signal.

HS is subject to both N-linked and O-linked sulfation along its backbone. As disclosed herein, modification of HS to remove some of the sulfation or to replace sulfate groups with acetyl groups, for example, can abrogate the ability of HS to activate TLR4. As such, factors that affect sulfation of HS can be useful to reduce TLR4 activity. For example, factors that remove sulfate groups from HS, or factors that inhibit or modulate sulfation of nascent HS molecules may be useful in the methods provided herein. Such factors might, for example, target sulfotransferases, (e.g., 6-0, 2-0, and 3-0 sulfotransferases) that may be responsible for sulfonating HS in vivo.

TLR activity can be monitored by a variety of methods. For example, TLR activity can be monitored by measuring the expression of a cytokine such as an interleukin or interleukin receptor (e.g., IL-1R, IL-1β, IL-4, IL-6, IL-6R, IL-7, IL-8, IL-10, IL-11, IL-12), tumor necrosis factor α or β (TNFα or β), osteoclast differentiation factor (ODF), or leptin, or a chemokine such as inducible protein 10 (IP-10), macrophage inflammatory protein 1α (MIP-1α), monocyte chemoattractant protein 1 (MCP-1), CC chemokine ligand 2 (CCL2), CC chemokine receptor, CXC chemokine LIX, or CC chemokine MIP-3α.

Other genes that are activated by TLR include cylooxygenase-2 (COX-2), inducible nitric oxide synthase (iNOS), extracellular signal-regulated kinase 1 (ERK1), ERK2, IL-1 receptor-associated kinase (IRAK), NFκB, activating protein-1 (AP-1), TLR2, secretory IL-1 receptor antagonist (sIL-1Ra), insulin-like growth factor binding protein-3 (IGFBP-3), vascular cell adhesion protein 1 (VCAM-1), p-selectin, β-integrin, vascular endothelial growth factor, β-nerve growth factor (NGF), lymphotoxin R, interferon regulatory factor 1 (IRF-1), mitochondrial hydroxymethylglutaryl-CoA synthase (HMG-CoA synthase), aldehyde dehydrogenase 2, neurotensin receptor 2, and protooncogenes such as c-Fos, Fos-B, Fra-2, Jun-B, Jun-D, or Egr-1. Surface markers that are expressed when TLR4 is activated include CD40, CD80, CD86, MHC class I, MHC class II, and CD25.

Expression of genes that are activated by TLR4 can be monitored by assessing mRNA or protein levels using standard molecular biology techniques, for example. Western blotting or immunoassays (e.g., ELISA) can be used to monitor protein production. Northern blotting, gene chip arrays, or polymerase chain reaction (PCR) techniques can be used to assess mRNA production. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase (RT) can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis Genetic Engineering News 12(9):1 (1992); Guatelli et al. (1990) Proc. Natl. Acad. Sci. USA, 87:1874-1878; and Weiss (1991) Science 254:1292.

Intracellular signaling pathways involving factors such as NFκB, AP1, and MAP (mitogen-activated protein) kinases (ERK, p38, JNK), Akt and phosphatidylinositol-3′-kinase (PI-3-K), protein kinase C, signal transducer and activator of transcription 1alpha (STAT1α), STAT1β, p38 (stress-activated protein kinase), Tollip, and c-Jun kinase also can be activated when TLR4 is activated. TLR4-stimulated activation of these pathways can be easily monitored by immunoblot or flow cytometric analysis using activation-state-specific antibodies directed against components of the monitored biochemical pathway.

In addition, small molecules such as PGE2 (prostaglandin E2), leukotriene B(4), or nitric oxide (NO) can be synthesized when TLR4 is activated. These end products can be detected using sandwich ELISA techniques or by colorometric chemical reactivity assays.

Compounds that inhibit TLR activity can be administered to a non-human subject, and bone density, bone strength, lean body mass, fat mass, or fat-free mass of the subject can be compared to that of a control subject (e.g., a corresponding non-human subject to which the test compound was not administered or to the baseline bone density or fat mass of the subject). Suitable non-human subjects include, for example, rodents such as rats and mice, rabbits, guinea pigs, farm animals such as pigs, turkeys, cows, sheep, goats, or chickens, or household pets such as dogs or cats.

A test compound can be administered to a subject by any route, including, without limitation, oral or parenteral routes of administration such as intravenous, intramuscular, intraperitoneal, subcutaneous, intrathecal, intraarterial, nasal, or pulmonary administration. A test compound can be formulated as, for example, a solution, suspension, or emulsion with pharmaceutically acceptable carriers or excipients suitable for the particular route of administration, including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives, flavorings, sugars, and other additives such as antimicrobials, antioxidants, chelating agents, inert gases, and the like also may be present.

For oral administration, tablets or capsules can be prepared by conventional means with pharmaceutically acceptable excipients such as binding agents (e.g., pregelatinized maize starch, polyvinylpyrrolidone or hydroxypropyl methylcellulose), fillers (e.g., lactose, microcrystalline cellulose or calcium hydrogen phosphate), lubricants (e.g. magnesium stearate, talc or silica), disintegrants (e.g., potato starch or sodium starch glycolate), or wetting agents (e.g., sodium lauryl sulfate). Tablets can be coated using methods known in the art. Preparations for oral administration also can be formulated to give controlled release of the compound. Nasal preparations can be presented in a liquid form or as a dry product. Nebulised aqueous suspensions or solutions can include carriers or excipients to adjust pH and/or tonicity.

The effect of a test compound on a non-human subject can be evaluated using a variety of methods. To monitor bone density, for example, markers of osteoblast differentiation can be assessed, for example, by monitoring expression of genes encoding osteoblastic markers such as alkaline phosphatase, osteocalcin, and type I collagen, or by examining levels of protein or protein activity. In addition, mineralization can be assessed as a marker of osteoblast differentiation. Bone mineral density or bone structure can be assessed using, for example, dual energy X-ray absorptiometry (DEXA), quantitative computed tomography, single photon absorptiometry, dual photon absorptiometry, or ultrasound techniques. In addition, bone strength may be monitored using an OsteoSonic device.

Fat mass and/or lean mass can be assessed using, for example, DEXA hydrodensitometry weighing (i.e., underwater weighing), anthropometry (i.e., skinfold measurements using calipers, for example), near infrared interactance (NIR), magnetic resonance imaging (MRI), total body electrical conductivity (TOBEC), air displacement (BOD POD), bioelectrical impedance (BIA), or computed tomography. The effect of a test compound on physical activity of a subject also can be monitored to get a sense of “exercise” changes and an initial sense of energy expenditure, since these may change in any animal that has differences in body fat and bone strength. The effect of a test compound on other characteristics related to metabolism also can be determined. These include, for example, characteristics related to food intake (e.g., appetite, taste/smell, pain with eating, satiation, parenteral nutrition, and enteral nutrition), characteristics related to digestion in the gastrointestinal tract (e.g., analyses of villous surfaces of gut, enzymes in gut, or bile salts), characteristics related to absorption, changes in caloric requirements, characteristics related to nutrient loss (e.g., through feces, hemorrhage, urine, fistulas, or loss through barriers such as the gastrointestinal tract, skin, or lung). Energy expenditure also can be monitored. For example, multi-directional motion can be monitored using a Mini Mitter device (Bend, Oreg.). Other characteristics related to energy expenditure that can be monitored include step/walking motion, heart rate, breathing, use of oxygen and output of carbon dioxide, photobeam monitoring, and charting of physical activity.

Methods of Using Compounds that Inhibit TLR Activity

Compounds that inhibit TLR activity and, in particular, TLR4 activity, can be used to reduce body fat, increase percent lean body mass, increase bone density, and/or reduce bone loss in a subject. In general, compounds that inhibit TLR activity can be formulated as described above and administered to a subject in an amount effective to reduce body fat, increase percent lean body mass, increase bone density, and/or reduce bone loss. Fat mass or bone density can be monitored in subjects after treatment using techniques described herein. Compounds that inhibit TLR activity can be administered to non-human subjects including farm animals such as pigs, turkeys, cows, chickens, goats, or sheep or household pets such as cats or dogs to increase percent lean body mass. In general, leaner animals live longer and, in addition, leaner farm animals are useful in meat production. Subjects being treated with TLR inhibitors may have an increased susceptibility to infections. Thus, in some embodiments, antibiotics can be administered prophylactically to animals receiving TLR inhibitors to prevent the development of infections.

Methods known in the art can be used to determine optimum dosages, dosing methodologies and repetition rates. Optimum dosages can vary depending on the relative potency of individual compounds, and can generally be estimated based on EC₅₀ found to be effective in in vitro and in vivo models. Typically, dosage is from 0.01 μg to 100 g per kg of body weight. TLR inhibitors may be given once or more daily, weekly, or even less often. Following successful treatment, it may be desirable to have the subject undergo maintenance therapy.

Compounds that inhibit TLR activity can be used to treat obesity (e.g., in humans or household pets). As described herein, animals containing mutant TLR4 and/or CD14 have less body fat and increased bone density than control animals containing wild type TLR4 or CD14 even though they are less physically active. Without being bound by a particular mechanism, obesity may be perpetuated and increased in an individual due to activation of Toll-like receptors, leading to inflammatory and non-inflammatory changes that impact fat and bone metabolism. Thus, TLR4 may be a master regulator of the inflammation that may cause obesity, and TLR4 inhibition or modulation therefore may be the key to successful treatment of obesity. In some embodiments, the usefulness of a TLR4 inhibitor for treating obesity and related disorders can be evaluated in comparison to, for example, a placebo or no treatment, estrogen replacement therapy, sibutramine (MERIDIA®), or orlistat (XENICAL®).

Compounds that decrease TLR activity, and in particular, TLR4 activity also can be used to increase bone density or reduce bone loss in subjects (e.g., humans). In some embodiments, compounds that decrease TLR activity are used to treat or prevent osteoporosis, a skeletal condition characterized by decreased density of normally mineralized bone, leading to an increased number of fractures. Primary osteoporosis, including post-menopausal, age-related, and idiopathic osteoporosis can be beneficially treated using this method. Secondary forms of osteoporosis caused by, for example, excessive alcohol intake, hypogonadism, hypercortisolism and hyperthyroidism also can be treated using this method. The effectiveness of a TLR4 inhibitor for treating osteoporosis and related disorders can be evaluated in comparison to, for example, a placebo or no treatment, estrogen replacement therapy, alendronate (FOSAMAX®), or risedronate (ACTONEL®).

### Articles of Manufacture

Inhibitors of TLR (e.g., TLR4) can be combined with packaging materials and sold as articles of manufacture or kits (e.g., for reducing body fat, increasing percent lean body mass, or for treatment of obesity or osteoporosis). Components and methods for producing articles of manufactures are well known. The articles of manufacture may combine one or more components described herein. In addition, the articles of manufacture may further include sterile water, pharmaceutical carriers, buffers, and/or other useful reagents (e.g., antibiotics). Instructions describing how such inhibitors can be used to reduce body fat, increase percent lean body mass, increase bone density, or treat obesity or osteoporosis may be included in such kits. The compositions may be provided in a pre-packaged form in quantities sufficient for a single administration or for multiple administrations in, for example, sealed ampoules, capsules, or cartridges.

The invention will be further described in the following examples, which do not limit the scope of the invention described in the claims.

### EXAMPLES

### Example 1

### Materials and Methods for Examples 2-7

Reagents and antibodies. Ultrapure HS and END-X endotoxin removal resin were obtained from Seikagaku (Falmouth, Mass.). LPS from Escherichia coli was from Sigma Aldrich (St. Louis, Mo.). Pancreatic elastase was from Calbiochem (La Jolla, Calif.). Anti-TLR4/MD2 antibody clone MTS510 was from e-Bioscience (San Diego, Calif.). Fluorescein isothiocyanate (FITC)-conjugated goat anti-rat IgG was from Southern Biotech (Birmingham, Ala.). Anti-phospho p38 mitogen activated protein kinase (MAPK), anti p38 MAPK and horseradish peroxidase-conjugated anti-rabbit antibodies were from Cell Signaling Technology (Beverly, Mass.). Rat anti-mouse CD86 was from Pharmingen (San Diego, Calif.). All materials used in cell culture were certified endotoxin free or were treated with endotoxin removal resin and tested by the Limulus amebocyte lysate assay gel clot method (Seikagaku) to assure absence of detectable endotoxin.

Plasmid construction. Total RNA was isolated from the murine macrophage cell line RAW 294.7 (ATCC, Manassas, Va.). This RNA was used to generate cDNA using the 1^{st} Strand cDNA Synthesis Kit (Roche, Indianapolis, Ind.) for RT-PCR (AMV) with oligo-dt primers (15 mer) and following reaction conditions: 25° C. for 10 minutes, 42° C. for 60 minutes, 99° C. for 5 minutes, and 4° C. for 5 minutes. The resulting pool of cDNA was used as a template to amplify TLR4, MD2 and CD14 coding sequences by PCR. Reactions were carried out using Expand High Fidelity polymerase (Roche) and the following conditions: 94° C. for 2 minutes followed by 25 cycles of 94° C. for 1 minute, 55° C. for 1 minute, and 68° C. for 3 minutes, finishing with 72° C. for 7 minutes. TLR4 was amplified using the following primers: TLR4 Forward 5′-CGCGGATCCAGGATGAT GCCTCCCTGGCTC-3′ (SEQ ID NO:1), and TLR4 Reverse 5′-GGCGGTACCTCAGG TCCAAGTTGCCGTTTC-3′ (SEQ ID NO:2). MD2 was amplified using MD2 Forward 5′-CCGGAATTCATCATGTTGCC-3′ (SEQ ID NO:3), and MD2 Reverse 5′-CCGGAA TTCCTAATTGACATCACG-3′ (SEQ ID NO:4). CD14 was amplified using CD14 Forward 5′-CCGGAATTCACCATGGAGCGTGTGCTTGGC-3′ (SEQ ID NO:5), and CD14 Reverse 5′-CCGGAATTCTTAAACAAAGAGGCGATCTCCTAG-3′ (SEQ ID NO:6). PCR products were digested with appropriate restriction enzymes and cloned into eukaryotic expression plasmids. TLR4 was cloned into pcDNA3.1 (Invitrogen, Carlsbad, Calif.). MD2 was cloned into pcDNA3.1/Hygro (Invitrogen). CD14 was cloned into pcDNA4/myc-His with zeocin resistance (Invitrogen). Cloned sequences were screened by restriction digestion for correct orientation. Nucleotide sequences were determined using the dideoxynucleotide reaction of Sanger and automated detection system (Mayo Clinic Molecular Biology Core Facility) and then compared to published sequences for the genes. A NFκB-firefly luciferase reporter plasmid was obtained from Dr. Carlos Paya (Paya et al., 1992). A control Renilla-luciferase reporter plasmid consisted of the Renilla-luciferase coding sequence under the control of the TK promoter (pTK-Renilla, Promega, Madison, Wis.).

Model ECM environment. Tissue culture plates were coated with ECM as follows. PAEC were seeded into 6-well (3×10⁵ cells/well), 24-well (5×10⁴ cells/well) or 100 mm (2×10⁵ cells) fibronectin-coated tissue culture plates (BD Biosciences, San Jose, Calif.) in DMEM (Invitrogen) containing 10% fetal bovine serum supplemented with penicillin and streptomycin and 4% w/v of Dextran 40. The cell cultures were incubated at 37° C. in 5% CO₂ humidified atmosphere for seven days and were supplemented with 50 mg/ml of ascorbic acid on day three and day five. After 7 days, endothelial cells were washed once with phosphate buffered saline (PBS) and lysed by exposure to 0.5% w/v Triton X-100 and 20 mM NH₄OH in PBS (pH 7.4) at 37° C. for 20 minutes (Bonifacino, 1998). The wells were then washed 4 times with PBS (pH 7.4) and inspected microscopically to ensure removal of the cells. Plates were used immediately or were stored in PBS (pH 7.4) with 50 mg/ml of gentamycin at 4° C.

Generation of ECM fragments. Tissue culture plates (100 mm) coated with ECM were treated with 1.0 ml elastase (0.1 U/ml) in PBS. The plates were sealed and incubated at 37° C. for 6 hours. The ECM fragments released from the plate by elastase were harvested, boiled for 30 minutes, and the total protein content was determined using bicinchoninic acid assay (Pierce, Rockford, Ill.). For some experiments, the harvested ECM fragments in PBS were adjusted to pH 6.0 using 0.1 N HCl and incubated with 0.5 mg recombinant human heparanase (see below) at 30° C. for 16 hours. The samples were readjusted to pH 7.5 and boiled for 30 minutes and the total protein concentration determined as above.

Cell culture and transfection. HEK 293 cells (ATCC) were maintained at 37° C. in 5% humidified CO₂ in DMEM containing 10% fetal bovine serum and penicillin and streptomycin. RAW 294.7 cells were maintained at 37° C. in 10% humidified CO₂ in DMEM containing 10% fetal bovine serum and penicillin and streptomycin.

HEK 293 cells were stably transfected with the TLR4 and MD2 or CD14 expression plasmids using Superfect (Qiagen, Valencia, Calif.) following the manufacturer's instructions. HEK 293 cells expressing TLR4 and MD2 or CD14 were obtained by culturing the transfected cells with appropriate antibiotic selection medium, and were cloned by limiting dilution in the selection medium. HEK 293 cells expressing TLR4, MD2 and CD14 were generated by transfecting HEK 293 cells that expressed TLR4 and MD2 with the CD14 expression plasmid, and selecting clones using appropriate antibiotic containing medium. Cell lines expressing TLR4 and MD2 and/or CD14 were then maintained in DMEM supplemented with 10% fetal bovine serum and the appropriate selection antibiotics. Control cell lines were transfected with empty expression vectors and incubated in selection conditions as described above. Selected clones were tested for cell surface expression of the TLR4/MD2 complex and CD14 by flow cytometry using monoclonal antibodies specific for the TLR4/MD2 complex or CD14.

Recombinant heparanase. Human heparanase cDNA was cloned as described (Dempsey et al. (2000) Glycobiology 10:467-475). Recombinant heparanase was produced using a baculoviral expression system and purified by affinity chromatography using heparin-agarose (McKenzie et al. (2003) Biochem. J. 373:423-35). The recombinant enzyme was dialyzed into PBS, pH 7.4, and concentrated to 57 mg/ml using Centricon 10,000 MWCO centrifugal concentrators, sterilized by filtration using 0.2 mm filters and stored at −70° C. until use.

Radiolabeling and depolymerization of HS. [³H]HS was prepared by reducing HS using [³H]BH₄ (Amersham) as described (Ihrcke et al. (1998) J. Cell. Physiol. 175:255-267). The radiolabeled product had a specific activity of 15 mCi/g.

HS or [³H]HS (20 mg/ml in water) was depolymerized by deaminative cleavage with nitrous acid (Conrad (2001) In Methods in Molecular Biology, R. V. Iozzo, ed. (Totowa, N.J., Humana Press), pp. 347-351) and then neutralized. Fragments of [³H]HS were separated using 10DG gel filtration columns (Biorad, Hercules, Calif.). Eluted fractions (0.25 ml) were collected and the [³H]HS was detected by scintillation counting. In some experiments, HS was depolymerized with recombinant human heparanase. Four micrograms of [³H]HS were incubated with 0.5 mg of recombinant human heparanase at 30° C. in 0.1 M sodium acetate, 0.1% bovine serum albumin buffer, pH 6.5. The reaction was stopped after 16 hours by increasing the pH to 8.0 and boiling for 30 minutes. The reaction was loaded onto a Hi-Trap Q column (Amersham-Pharmacia, Piscataway, N.J.) and the HS fragments were eluted with a linear gradient of NaCl (0 to 1 M). Radioactivity in the eluted fractions (0.5 ml) was detected by scintillation counting.

NFκB-luciferase reporter assays. Activation of NFκB was measured using a NFκB-luciferase reporter assay. HEK 293 cell lines stably expressing TLR4, MD2 and/or CD14, or control cells were seeded into 24 well tissue culture plates (2×10⁵ cells/well) in 1.0 ml DMEM containing 10% fetal bovine serum and penicillin and streptomycin. The cells were allowed to adhere to the culture wells at 37° C. overnight and were then transfected with 0.1 mg pTK Renilla-luciferase and 0.1 mg NFκB-firefly-luciferase using Superfect Transfection Reagent (Qiagen). Following transfection, the cells were washed once with phosphate buffered saline and cultured for 24 hours at 37° C. in 1.0 ml DMEM containing 0.5% fetal bovine serum. After various treatments, the culture medium was aspirated and the cells were washed once with PBS. The cells were lysed in 150 ml Passive Lysis Buffer (Promega) with rocking at room temperature for 15 minutes. Renilla- and Firefly-luciferase were assayed simultaneously using the Dual-Luciferase Reporter Assay System (Promega) and a TD-20/20 luminometer (Turner Designs, Sunnyvale, Calif.). Activation of NFκB was reported as a ratio of the firefly luciferase activity to the constitutively expressed Renilla luciferase internal control, and is the mean of triplicate wells.

Animals. TLR4-deficient C57BL/10ScN mice, which have a deletion in chromosome 4 that encompasses the TLR4 gene, were obtained from The National Cancer Institute, Bethesda, Md. C57BL/10SnJ mice, which have wild type TLR4 and are congenic with C57BL/10ScN were from the Jackson Laboratory, Bar Harbor, Me.

Surgical Procedures and Immunohistochemistry. Mice were anesthetized and the spleen was directly visualized through an incision in the lateral abdominal wall. Particular substances in 50 ml of PBS were injected into the spleen. Spleens were harvested after 12 hours and sections were frozen in liquid nitrogen. Tissue sections were prepared and stained as described in (Dempsey et al. supra) with the following modifications. Secondary detection antibodies were diluted in M.O.M. diluent (Vector Laboratories, Burlingame, Calif.) and preabsorbed with mouse serum (Jackson ImmunoResearch Laboratories, West Grove, Pa.). Fluorescent images were converted to grayscale using SPOT software (Diagnostic Instruments, Sterling Heights, Mich.).

### Example 2

### TLR4 Activation by HS

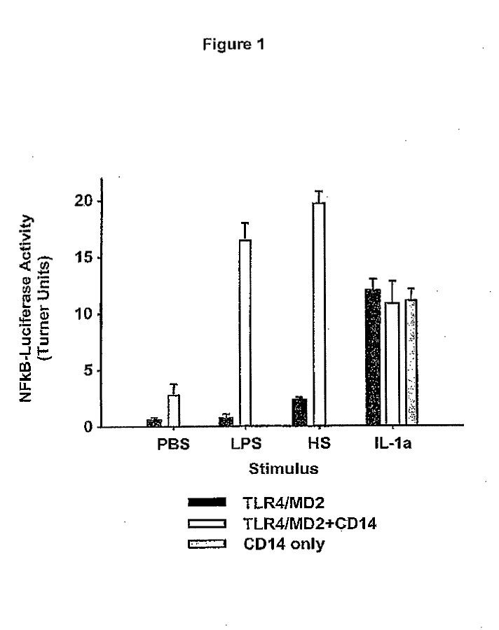

To determine whether and under what conditions HS stimulates TLR4, various combinations of TLR4, MD2 and CD14, components of the putative TLR4 receptor complex, were stably expressed in HEK 293 cells and the effect of HS on TLR4 signaling in the transfected cells was examined (FIG. 1). The HEK 293 cells expressing receptor components were transiently transfected with a NFκB-firefly luciferase reporter construct along with a Renilla-luciferase internal control reporter construct. HEK 293 cells that expressed TLR4, MD2, and CD14 were activated by HS or by LPS (FIG. 1), while HEK 293 cells lacking TLR4 and MD2 or CD14 did not respond. HEK 293 cells lacking TLR4 did have the capacity to activate NFκB, as cells lacking TLR4 or CD14 were activated by recombinant human IL-1α, which uses the same intracellular apparatus as TLR4 to activate NFκB (Magor and Magor (2001) Dev. Comp. Immunol. 25:651-682). The HEK 293 cell clone that expresses TLR4, MD2, and CD14 is referred to as HEK/TLR4(+) in the experiments that follow.

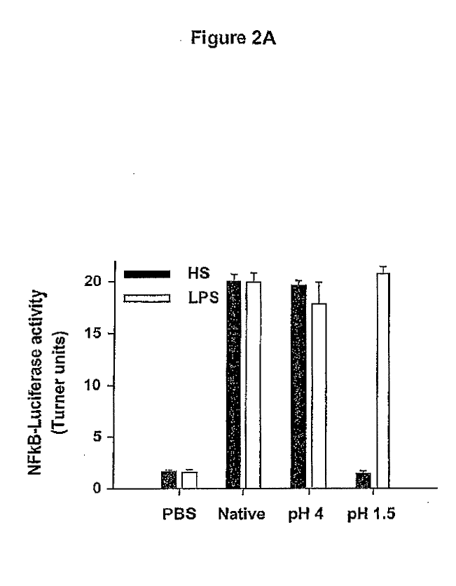

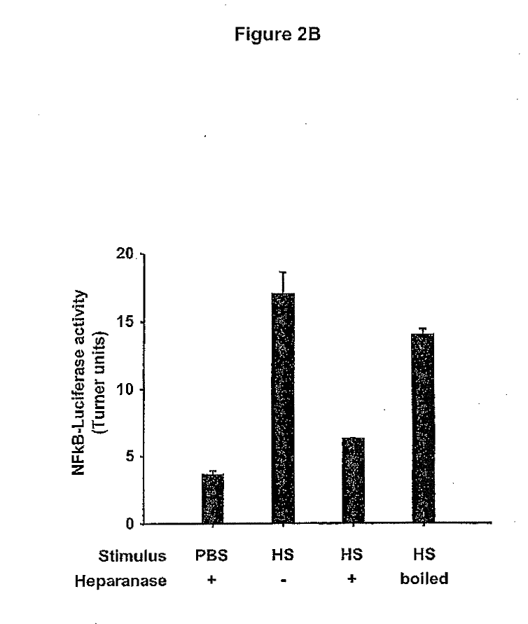

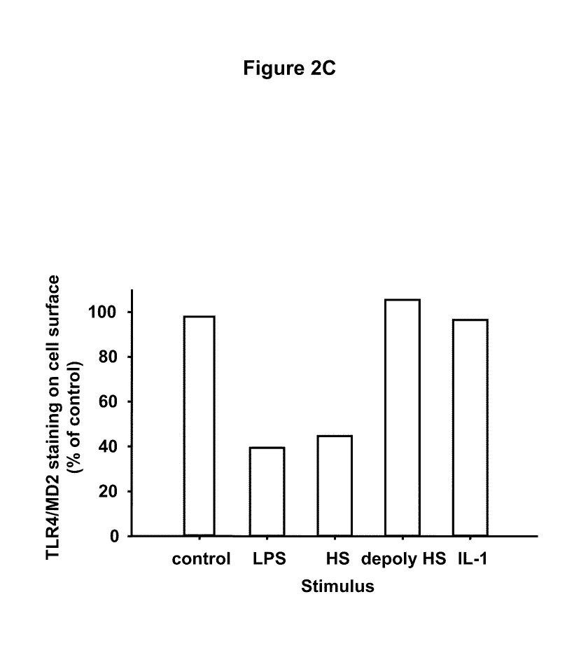

To confirm that TLR4 was stimulated by HS rather than by some other substance in the medium or matrix, the effect of depolymerized HS was tested on HEK/TLR4(+) cells. HS that was depolymerized with nitrous acid at pH 4.0, which cleaves at unmodified glucosamine residues (Conrad supra), stimulated HEK/TLR4(+) cells fully (FIG. 2A). However, HS that was depolymerized with nitrous acid at pH 1.5, which cleaves at sulfated glucosamine residues, did not stimulate HEK/TLR4(+) cells. Under both conditions, nitrous acid did not affect the ability of LPS to stimulate TLR4 (FIG. 2A). HS that was depolymerized with heparanase, which cleaves adjacent to highly sulfated regions of the polysaccharide (Okada et al. (2002) J. Biol. Chem. 277:42488-42495), abolished two thirds of the agonist activity (FIG. 2B). These results demonstrate that TLR4 is activated by HS.

To determine whether the effect of HS occurred through specific action on TLR4, surface expression of the TLR4/MD2 complex on cells treated with HS was examined. Treatment of HEK/TLR4(+) cells with HS caused a 60% decrease in surface expression of TLR4/MD2, a decrease similar to that induced by LPS (FIG. 2C). HS that had been depolymerized with nitrous acid at pH 1.5 did not decrease TLR4/MD2 surface expression. Recombinant human IL-1α, which activated NFκB via the related IL-1 receptor (FIG. 1), did not change surface expression of TLR4/MD2. Thus, HEK/TLR4(+) cells responded to HS by activating NFκB and mobilizing receptor complexes.

### Example 3

### ECM Inhibits TLR4 Signaling

To determine whether TLR4 interacts with HS proteoglycans under quiescent conditions, HEK/TLR4(+) cells were cultured on six well plates coated with PAEC ECM, which is rich in HS proteoglycans. PAEC were cultured to confluence on fibronectin-coated plates and then removed, leaving the ECM. Control HEK/TLR4(+) cells were cultured on plates coated with fibronectin alone. HEK 293 cells transfected with NFκB- and control-luciferase reporter genes but not expressing TLR4 and MD2 also were used as controls.

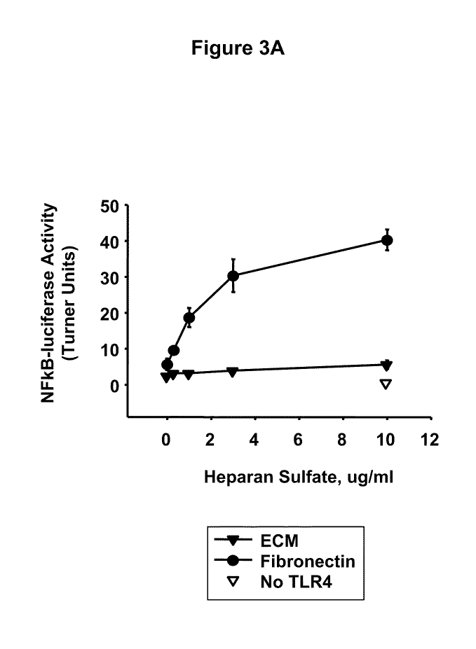

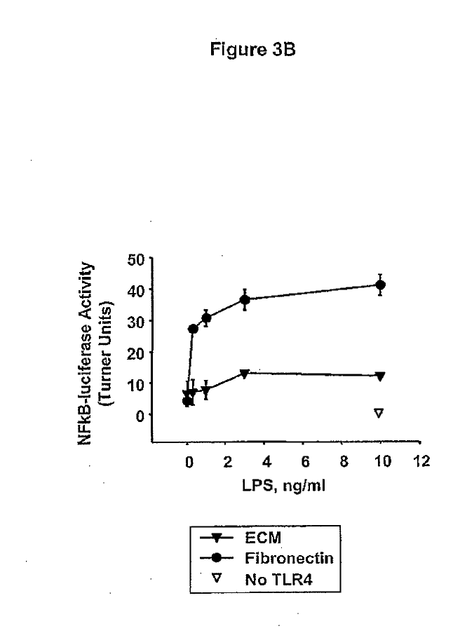

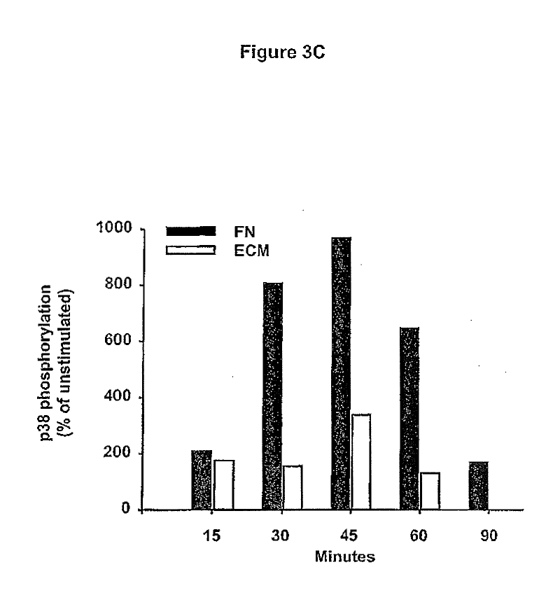

HEK/TLR4(+) cells cultured on the PAEC ECM had a low baseline level of NFκB-luciferase activity, similar to HEK/TLR4(+) cells cultured on fibronectin (FIG. 3A). HEK/TLR4(+) cells cultured on the ECM responded minimally to stimulation with HS (FIG. 3A) or with LPS (FIG. 3B). In contrast, HEK/TLR4(+) cells cultured on fibronectin responded fully. Expression of TLR4/MD2 was not different in cells cultured on ECM or fibronectin Inhibition of TLR4 was not unique to HEK/TLR4(+) cells, as RAW 264.7 macrophages, which naturally express TLR4, had a profoundly blunted response to HS when cultured in ECM. Activation of p38 MAP kinase by HS was reduced by about 60% when RAW macrophages were cultured on ECM rather than on fibronectin (FIG. 3C). Thus, ECM suppresses signaling delivered through TLR4.

To determine whether suppression of TLR4 signaling by ECM occurred at the level of TLR4 complexes or the intracellular signaling apparatus, activation of NFκB signaling by IL-1α and TNFα was measured. These cytokines use the same intracellular components as TLR4 (Magor and Magor (2001) Dev. Comp. Immunol. 25:651-682). Cells were cultured on fibronectin- or ECM-coated plates and transfected with NFκB- and control-luciferase reporter plasmids. The transfected cells were treated with 10 ng/ml of recombinant human IL-1αor TNF-αfor 6 hours, and luciferase activity was measured. Reporter activity in cells cultured on EMC was expressed as a percentage of the activity obtained in control cells grown on fibronectin.

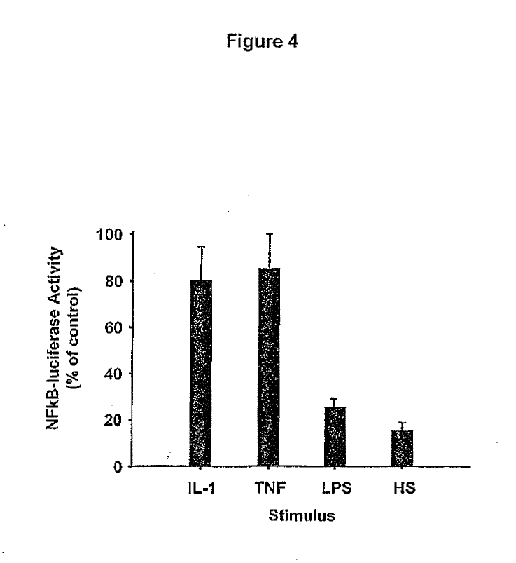

Although the absolute degree of signaling in response to IL-1αor TNFα varied, the NFκB signal was approximately 20% lower in cells cultured in ECM compared to cells cultured on fibronectin (FIG. 4). This lower level of NFκB signaling was in contrast to the 80%-90% decrease observed when TLR4 agonists (LPS or HS) were used (FIGS. 3A and 3B). These results indicate that ECM acts on TLR4 complexes and minimally or not at all on the intracellular signaling apparatus.

### Example 4

### Elastase Prevents Inhibition of TLR4 Signaling by ECM

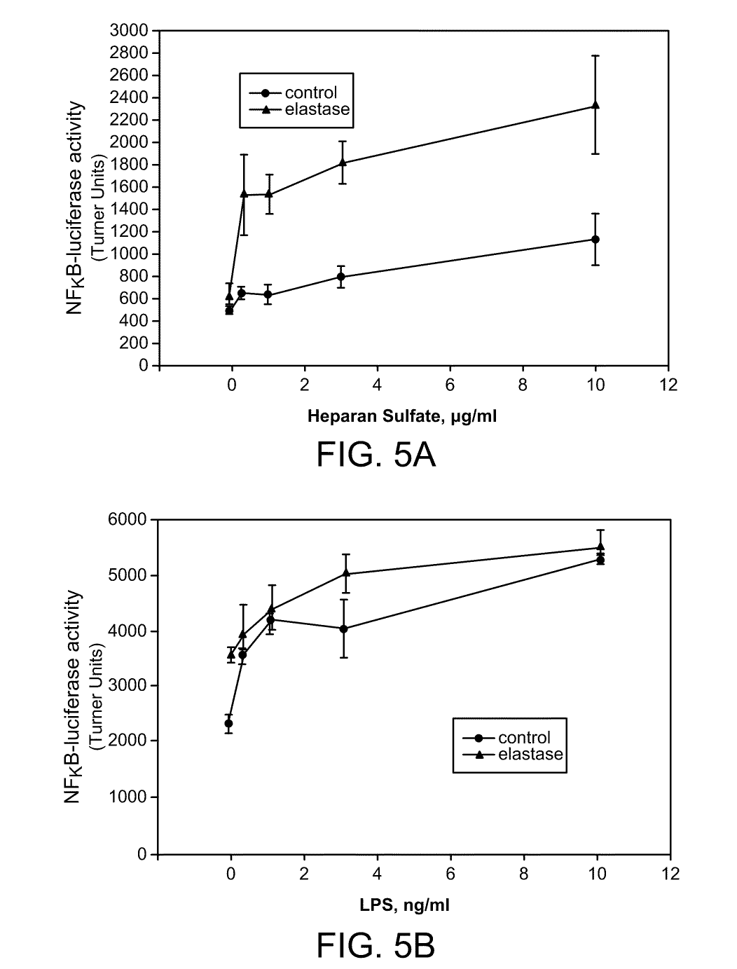

If ECM inhibits TLR4 activation, an important question would be how the receptor complex is relieved of this inhibition so that inflammation or resistance to infection can be mounted. It is possible that whereas TLR4-expressing cells might be refractory to inadvertent signaling in healthy or unperturbed tissues, those cells might respond vigorously in tissue injury where ECM is cleaved by proteases. To test this idea, HEK/TLR4(+) cells were cultured on plates coated with PAEC ECM and then transiently transfected with the NFκB reporter plasmid described in Example 1. After 6 hours, the cells were pre-treated with elastase, a protease released by neutrophils that cleaves ECM proteins including HS proteoglycans (Belaaouaj et al. (1998) Nat. Med. 4:615-618). Control cells were not pre-treated with elastase. The cells were then stimulated with LPS or HS as above. As shown in FIGS. 5A and 5B, pre-treatment with elastase and treatment with HS or LPS resulted in activation of reporter expression that was greater than the activation observed in the untreated control cells.

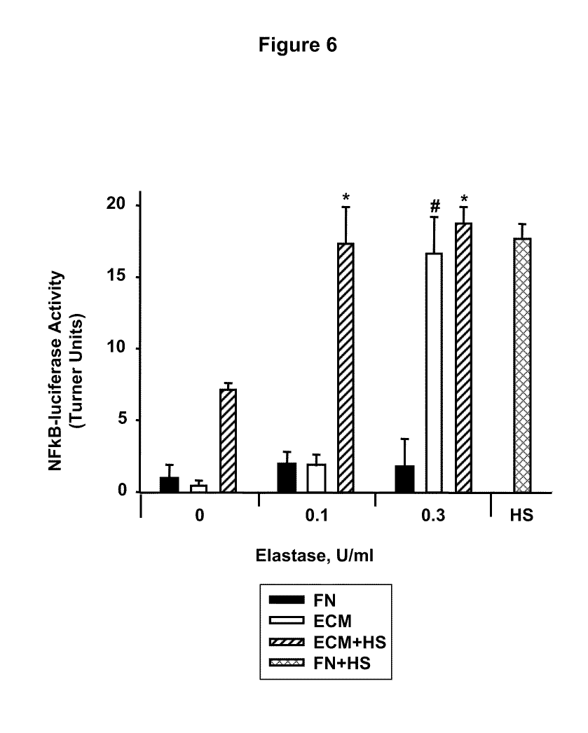

HEK/TLR4(+) cells cultured in ECM did not respond to a low concentration of elastase (0.1 U/ml; FIG. 6). However, cells treated with this low (non-stimulatory) concentration of elastase responded to HS with a significant 2 fold greater activation than cells not treated with elastase. HEK/TLR4(+) cells cultured on ECM treated with a higher concentration of elastase (0.3 U/ml) responded even without addition of HS. HEK/TLR4(+) cells on fibronectin did not respond to either concentration of elastase. These results indicate that inhibition of TLR4 by ECM is relieved by elastase.

### Example 5

### ECM from Different Cell Types have Different Effects on TLR4 Signaling

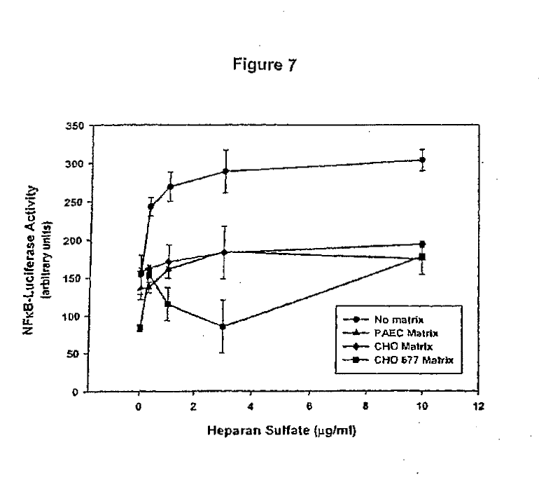

HEK 293 cells stably expressing TLR4 were cultured on plates coated with PAEC ECM, ECM from Chinese hamster ovary (CHO) cells, or ECM from CHO 667 cells, which are defective in HS synthesis. Control cells were cultured on uncoated plates. As above, cells were transiently transfected with the NFκB reporter plasmid and incubated for 6 hours before measurement of luciferase activity. These experiments revealed that all three types of cellular matrices were able to inhibit TLR4-mediated activation of the reporter plasmid (FIG. 7), indicating that proteoglycans in addition to HS proteoglycans also may contribute to the observed inhibitory effect.

### Example 6

### Cleaved Components of ECM Activate TLR4 Signaling

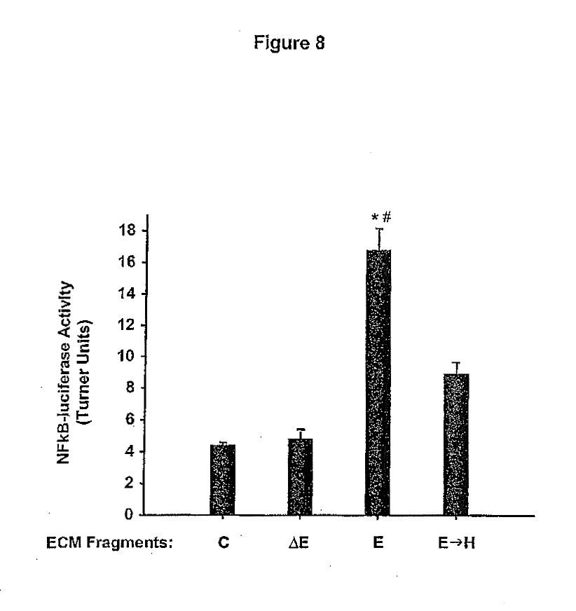

Experiments were conducted to determine the extent to which HS in ECM activates TLR4 signaling in response to elastase. As shown in FIG. 8, ECM fragments that were released by elastase treatment were able to activate HEK/TLR4(+) cells cultured in untreated plates, while ECM fragments that were generated using inactivated (boiled) elastase did not stimulate the cells. Treatment of the ECM fragments with heparanase decreased the HS content of the fragments by approximately 50%, and also diminished by 50% the ability of the fragments to activate the NFκB-luciferase reporter in HEK/TLR4(+) cells (FIG. 8). Thus, HS contributes significantly to TLR4 stimulation by ECM fragments generated by elastase.

### Example 7

### ECM Cleaved In Vivo Activates TLR4

To determine whether elastase activity on ECM activates TLR4 responses in living tissues, elastase was injected into the spleens of mice. Such treatment typically causes rapid shedding of HS proteoglycans from the tissue, especially from blood vessels (Johnson et al. (2004) J. Immunol. (Cutting Edge) 172:20-24). The spleens were removed and examined for expression of CD86, a protein expressed in response to TLR4 signaling (Kaisho and Akira (2002) Biochim. Biophys. Acta 1589:1-13). Injection of LPS or HS modestly increased expression of CD86 in the spleen. In contrast, the expression of CD86 observed in leukocytes detached from ECM and stimulated with LPS or HS is considerably heightened (Johnson et al. supra). Injection of elastase profoundly increased expression of CD86 in the spleen. The changes in CD86 expression induced by elastase required TLR4, as the increase in CD86 was not observed when elastase was injected into spleens of mice lacking TLR4 function. Thus, the ECM may limit TLR4 signaling in vivo, and cleavage of ECM can trigger TLR4 signaling in living tissues.

### Example 8

### Modification of HS Sulfation Abrogates Signaling

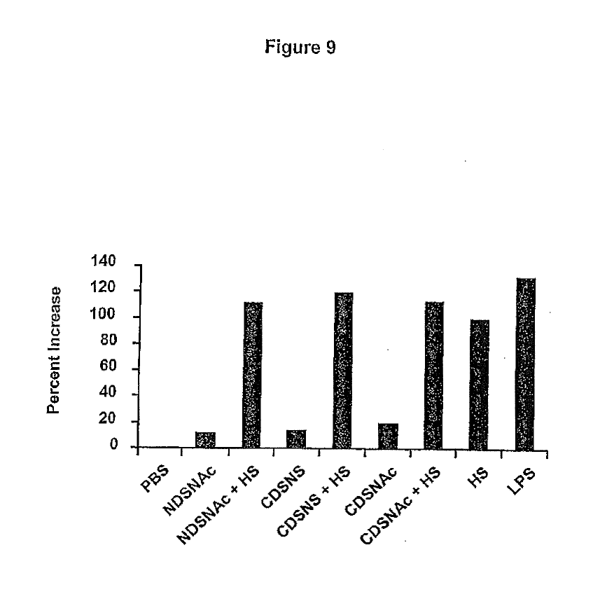

HS is sulfated extensively along its sugar backbone, with both N-linked and O-linked sulfation. The pattern of sulfation is non-random, but is highly variable and distinct from the pattern of heparin sulfation. Soluble HS (Seikagaku) was modified by N-desulfation followed by N-acetylation (NDSNAc), by complete desulfation (both N- and O-sulfation removed) followed by N-sulfation (CDSNS), or by complete desulfation followed by N-acetylation (CDSNAc). Modified HS (100 μg/ml final concentration), unmodified HS (10 μg/ml final concentration), combinations of these, LPS (10 ng/ml final concentration), or PBS vehicle alone was added to cultures of immature murine dendritic cells. After 24 hours, the cells were washed and stained for CD40 surface expression, and immunofluorescence was measured by flow cytometry. FIG. 9 shows dendritic cell maturation in response to stimulation with TLR4 agonists, measured as the percent increase in CD40 expression. Modified HS was not stimulatory to the cells, even at 10 times the dose of unmodified HS.

### Example 9

### Materials and Methods for Examples 10-14

Reagents and Antibodies. Unconjugated monoclonal HepSS-1 (anti-HS) was from US Biological (Swampscott, Mass.). Limulus anti-LPS factor (LALF) was from Associates of Cape Cod (Woods Hole, Mass.). CpG sequence ODN1826 (Askew et al. (2000) J. Immunol. 165:6889) phosphorothioate-modified single-stranded oligonucleotide was synthesized, then quantitated spectrophotometrically. Bovine kidney-derived HS (super special grade), and chondroitin sulfate B were purchased from Seikagaku (Falmouth, Mass.). Escherichia coli-derived LPS B4:0111, D-galactosamine, type IV porcine pancreatic elastase, zymosan A, and amebocyte lysate from Limulus polyphemus were from Sigma-Aldrich (St. Louis, Mo.). Elastase inhibitor-1 was obtained from Calbiochem (La Jolla, Calif.). Pharmaceutical grade heparin was from Elkins-Sinn (Chemy Hill, N.J.).

Animals. Mice used in these studies included TLR4-deficient C57BL/10ScNCr (National Cancer Institute, Bethesda, Md.), TLR4-mutant C3H/HeJ, and their TLR4 wild-type control strains C57BL/10SnJ and C3H/HeSnJ, respectively (The Jackson Laboratory, Bar Harbor, Me.).

Enzyme Purification. Enzymes were purified before use by passage over a polymyxin B cross-linked 6% agarose column (Pierce Biotechnology, Rockford, Ill.). Fractions of each enzyme preparation were boiled and tested to have only trace LPS contamination (<1% of limiting concentration needed to evoke responses) by Limulus amebocyte lysate assay gel clot method (Seikagaku). Human platelet heparanase was purified as previously described (Ihrcke et al. supra) and then dialyzed into PBS to a final concentration of 3.4 mg/ml and stored at −80° C. until use. Heparanase activity was measured as previously described (Ihrcke et al. supra) to be 0.19 μg of HS released per microgram of heparanase per hour.

Cell Isolation and Culture. Dendritic cells were generated from murine bone marrow cultures as previously described (Kodaira et al. (2000) J. Immunol. 165:1599). At day 6 or 7 of culture, nonadherent cells and loosely adherent proliferating dendritic cell aggregates were harvested for analysis or stimulation. PAEC were cultured to confluence and their identity was confirmed as previously described (Ryan and Maxwell (1986) J. Tissue Cult. Methods 10:3).

Cell Culture Stimulation. Dendritic cells (2×10⁶ per ml) cocultured with confluent monolayers of PAEC in 96-well plates were stimulated with 10 μg/ml chondroitin sulfate, 500 ng/ml CpG DNA, 10 ng/ml LPS, 10 μg/ml HS, 50 μg/ml zymosan, or PBS, unless otherwise indicated. To control for purity, agonists were pretreated with END-X B15, or mixed with Limulus anti-LPS factor before stimulation of cells, as indicated. In some experiments, agonists were boiled before use for 60 minutes at 100° C.

Cytokine Quantification. Age-matched female mice were injected in the peritoneum with 0.5 U of elastase, 5 mg of HS, 200 U of heparin, 150 μg of CpG DNA, or PBS with a total volume of 250 μl. One hour and 3 hours after injection, 100-μl blood samples were collected from the tail vein. Cell supernatants and serum samples were immediately frozen at −20° C. until analysis. Concentrations of TNF-α were analyzed by enzyme-linked sandwich ELISA (R&D Systems, Minneapolis, Minn.).

Immunopathology. After intraperitoneal (i.p.) injection of ketamine and xylazine, murine spleens were directly visualized through an incision in the lateral abdominal wall and injected with 100 μl of PBS containing 0.1 U of elastase or PBS alone. Five hours later, the spleen was harvested and pieces snap-frozen. Tissue sections were prepared and stained as previously described (Dempsey et al. supra) with the several modifications. In particular, secondary and tertiary antibodies were mouse serum (Jackson ImmunoResearch Laboratories, West Grove, Pa.) preabsorbed and diluted in M.O.M. diluent (Vector Laboratories, Burlingame, Calif.). Fluorescent images were converted to grayscale using SPOT software (Diagnostic Instruments, Sterling Heights, Mich.).

Experimental shock model. Age- and sex-matched mice were injected with 5 mg of HS, 5 μg of LPS, 5 μg of Limulus anti-LPS factor, 5 mg of chondroitin sulfate, 200 U (−5 mg) heparin, 150 μg of CpG DNA, 1.5 U of elastase, or PBS mixed with 20 mg of D-galactosamine in a total volume of 500 μl of PBS by i.p. injection as previously described (Franks et al. (1991) Infect. Immun. 59:2609). Concentrations of HS and elastase were calculated by weight of lyophilized powder, and the doses used were near the LD₅₀ based on dose-response experiments. In some experiments, the agonist was mixed with 5 or 20 μg of Limulus anti-LPS factor before injection. For some injections, enzymes were boiled at 100° C. for 60 minutes and vortexed vigorously, or preincubated with elastase inhibitor-1 for 4 hours at room temperature and then mixed with D-galactosamine before injection. Mice were monitored every hour for 48 hours and then euthanized.

### Example 10

### Soluble HS Induces Responses in TLR4 Wild-Type and Mutant Mice

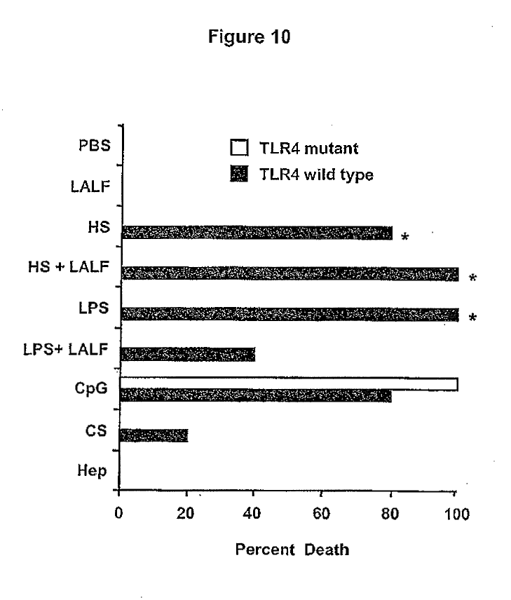

During sepsis, Gram-negative bacteria shed LPS, which activates TLR4 on cells that then release inflammatory cytokines mediating systemic inflammation and death (Beutler (2000) Curr. Opin. Immunol. 12:20). To determine whether soluble HS can induce a model of systemic inflammatory response syndrome (SIRS) via activation of TLR4 in mice, HS was administered by i.p. injection to TLR4 wild type and mutant mice. This mouse model system has been used to study shock and a sepsis-like syndrome in response to microbial toxins (Galanos et al. (1979) Proc. Natl. Acad. Sci. USA 76:5939). Wild-type mice injected with LPS died, as did eighty percent of TLR4 wild-type mice injected with HS (FIG. 10). In contrast, no TLR4-mutant mice injected with HS died. The TLR4-mutant mice were capable of undergoing SIRS, as the condition could be induced in these mice by administration of CpG DNA, a TLR9 agonist (Hemmi et al. (2000) Nature 408:740). The response was specific for HS, because mice injected with heparin (which is structurally related to HS but does not stimulate TLR4) survived. In addition, all but one mouse injected with chondroitin sulfate, which has the same charge density as HS, also survived. Moreover, the observed responses were not due to contamination of HS by LPS, since the HS used was not contaminated with LPS. In addition, the Limulus anti-LPS factor had no impact on the ability of HS, but greatly diminished the ability of LPS to induce death in treated mice (FIG. 10).

### Example 11

### Elastase-Induced Responses in TLR4 Wild Type and Mutant Mice

To determine whether endogenous stores of HS could trigger SIRS, pancreatic elastase was injected into the peritoneal cavity of mice. As described above, for example, elastase cleaves HS from cell surfaces and extracellular matrices in vitro, thus liberating endogenous HS. Fifty percent of wild-type mice injected with elastase died, whereas no TLR4 mutant mice died, suggesting that injection of elastase leads to activation of TLR4 and thus to death (Table 1). The elastase solution was not contaminated with LPS, as it was passed through a polymyxin B column and confirmed to contain <1% of a limiting dose of LPS by Limulus amebocyte lysate assay. Boiling elastase, which does not inactivate LPS but does denature elastase, eradicated the response (Table 1). Moreover, elastase inhibitor-1, a specific inhibitor of pancreatic elastase, reduced the death rate by 50%. These results indicate that the enzymatic activity of elastase is required for activation of TLR4.

TABLE 1
Percent death after injection of elastase in SIRS shock model

| | TLR4 Mutant (%) | TLR4 Wild Type (%) |
|---|---|---|
| Elastase | 0 | 50 |
| Boiled Elastase | 0 | 0 |

### Example 12

### TNF-α Secretion in Response to Degradation of HS Proteoglycan

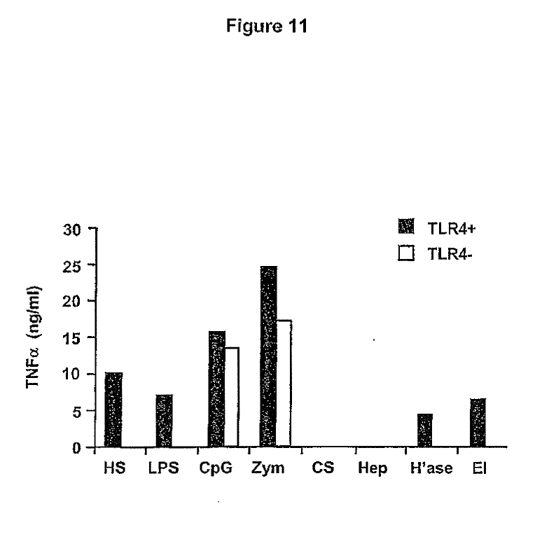

Experiments were conducted to determine whether specific shedding of HS induced by the action of heparanase, an endoglycosidase that specifically cleaves HS (Bame (2001) Glycobiology 11:91R), would also trigger TLR4 activation. Heparanase purified from human platelets (Gonzalez-Stawinski et al. (1999) Biochim. Biophys. Acta 1429:431) was added to 24-hour-old cocultures of PAEC and murine APCs that were TLR4-positive or -negative, and then assayed TNF-α. The heparanase was passed over polymyxin B columns and confirmed by Limulus amebocyte lysate assay to lack LPS. Aortic endothelial cells express an abundance of HS proteoglycans (Platt et al. (1990) J. Exp. Med. 171:1363) that are released into solution by elastase (Klebanoff et al. (1993) Am. J. Pathol. 143:907) and heparanase (Matzner et al. (1985) J. Clin. Invest. 76:1306). In response to soluble HS, elastase, or heparanase, the APC secreted TNF-α in a TLR4-dependent manner and responded to control stimulants as expected (FIG. 11).

### Example 13

### Effects of HS and Elastase on Serum TNF-α Levels in TLR4 Wild Type and Mutant Mice

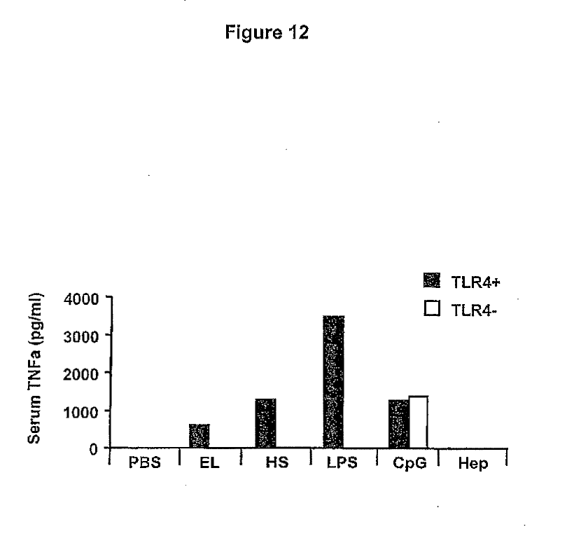

To determine whether HS or enzymes that release HS lead to high serum levels of TNF-α via TLR4, HS, elastase, or PBS was administered to wild type and mutant mice, and serum TNF-α was measured after 1 hour and 3 hours. Wild-type mice treated with HS or elastase had high serum levels of TNF-α 1 hour after treatment (FIG. 12). In contrast, TNF-α was not detectable in the TLR4-mutant mice even after 3 hours. TLR4-mutant mice did respond to the TLR9 agonist CpG DNA, used as a positive control, by producing TNF-α (FIG. 12).

### Example 14

### Effects of Elastase on Endogenous HS Proteoglycan In Vivo

To determine whether elastase liberates HS in vivo, spleen tissues were harvested from mice that had been injected intrasplenically with the enzyme, and the tissues were tested for the presence of HS. Mice injected with pancreatic elastase lost HS from blood vessels at the injection site within 5 hours of injection. Thus, pancreatic elastase can induce loss of HS proteoglycan from tissues in vivo.

### Example 15

### Body Mass of Mice Lacking TLR4

Lean body mass, body mass, percent body fat, and fat body mass in female mice lacking functional TLR4 (C3H/HeJ; Jackson Labs) were compared to the same characteristics in age and sex matched control mice having functional TLR4 (C3H/HeSnJ; Jackson Labs). The results are presented in Table 2. Mice lacking functional TLR4 (C3H/HeJ) rarely gained more than 17% fat body mass, and the body fat that they did possess had a normal distribution. The C3H/HeJ mice had athletic bodies even though they were housed in cages. This is in contrast to the control mice, which gained significantly more fat body mass. Lean body mass was less affected by the mutation in TLR4 than fat body mass.

These findings were confirmed by comparing a separate strain of mice with a different TLR4 mutation to its wild-type control strain. The second strain of mice, C57B1/10ScNCr, contains a naturally occurring TLR deletion (a recessive deletion of the entire gene). These mice were purchased from the National Cancer Institute. As shown in Table 3, the C57B1/10ScNCr mice also were significantly leaner than wild-type controls (C57B1/10SnJ; Jackson Labs).

TABLE 2
TLR4 Mutant (C3H/HeJ)

| | Age | | | | | | |
|---|---|---|---|---|---|---|---|
| Measurement | (wks) | N | P value | Average | | St. Dev | % of WT |
| Lean Mass | 8 | 5 | 0.28 | 14.26 | g | 1.08 | 93.2% |
| Lean Mass | 10 | 8-9 | 0.0021 | 13.53 | g | 1.22 | 90.0% |
| Lean Mass | 12 | 5-6 | 0.0946 | 15.37 | g | 1.04 | 92.2% |
| Lean Mass | 12-B* | 10 | 3.49 × 10⁻⁵ | 13.49 | g | 1.85 | 78.8% |
| Lean Mass | 24 | 15 | 0.0004 | 16.62 | g | 1.52 | 87.8% |
| Lean Mass | 31 | 14 | 3.7 × 10⁻⁸ | 16.01 | g | 1.13 | 82.6% |
| Body Mass | 8 | 5 | 0.11 | 16.20 | g | 1.24 | 85.3% |
| Body Mass | 10 | 8-9 | 0.0043 | 15.32 | g | 1.42 | 86.4% |
| Body Mass | 12 | 5-6 | 0.0205 | 18.47 | g | 1.40 | 77.5% |
| Body Mass | 12-B | 10 | 1 × 10⁻⁶ | 15.27 | g | 2.19 | 70.4% |
| Body Mass | 24 | 15 | 1 × 10⁻⁷ | 19.10 | g | 1.83 | 71.0% |
| Body Mass | 31 | 14 | 1.23 × 10⁻¹² | 19.51 | g | 1.94 | 62.3% |
| Fat Mass | 8 | 5 | 0.038 | 1.96 | g | 0.18 | 53.6% |
| Fat Mass | 10 | 8-9 | 0.00081 | 1.78 | g | 0.29 | 66.3% |
| Fat Mass | 12 | 5-6 | 0.00978 | 3.10 | g | 0.46 | 69.2% |
| Fat Mass | 12-B | 10 | 4 × 10⁻⁷ | 1.78 | g | 0.44 | 38.9% |
| Fat Mass | 24 | 15 | 5 × 10⁻⁹ | 2.62 | g | 0.54 | 32.9% |
| Fat Mass | 31 | 14 | 1.70 × 10⁻¹³ | 3.49 | g | 0.99 | 29.2% |
| % Fat | 8 | 5 | 0.011 | 11.92% | | 0.32 | 63.4% |
| % Fat | 10 | 8-9 | 0.0026 | 11.57% | | 1.14 | 76.7% |
| % Fat | 12 | 5-6 | 0.0180 | 16.75% | | 1.53 | 79.3% |
| % Fat | 12 | 10-B | 1 × 10⁻⁶ | 11.53% | | 1.85 | 55.4% |
| % Fat | 24 | 15 | 2 × 10⁻¹² | 13.30% | | 1.36 | 45.8% |
| % Fat | 31 | 14 | 1.11 × 10⁻¹⁵ | 17.28% | | 2.93 | 45.6% |
| Bone Density | 8 | 5 | 0.21 | 0.0467 | g/cm² | 0.0015 | 97.07% |
| Bone Density | 10 | 8-9 | 0.58 | 0.0454 | g/cm² | 0.0023 | 98.77% |
| Bone Density | 24 | 15 | 7.67 × 10⁻⁴ | 0.0596 | g/cm² | 0.0024 | 105.08% |
| Bone Density | 31 | 14 | 1.59 × 10⁻⁵ | 0.0620 | g/cm² | 0.0022 | 106.68% |
| Bone Calcium | 8 | 5 | 0.71 | 0.3600 | g | 0.0260 | 101.35% |
| Bone Calcium | 10 | 8-9 | 0.63 | 0.3342 | g | 0.0307 | 102.01% |
| Bone Calcium | 24 | 15 | 1.71 × 10⁻⁸ | 0.5254 | g | 0.0286 | 117.21% |
| Bone Calcium | 31 | 14 | 0.00015 | 0.5211 | g | 0.0336 | 113.20% |
| Bone Area | 8 | 5 | 0.24 | 7.7040 | cm² | 0.3913 | 128.01% |
| Bone Area | 10 | 8-9 | 0.21 | 7.3500 | cm² | 0.3347 | 103.14% |
| Bone Area | 24 | 15 | 2.62 × 10⁻⁷ | 8.8226 | cm² | 0.3992 | 111.80% |
| Bone Area | 31 | 14 | 0.0124 | 8.4507 | cm² | 0.4214 | 106.65% |

*B = live in the Barrier facility (sterile)

TABLE 3
TLR4 Deleted (C57B1/10ScNCr)

| Measurement | Age (wks) | N | P value | Average | | St. Dev | % of WT |
|---|---|---|---|---|---|---|---|
| Lean Mass | 6 | 10 | 0.0887 | 14.5875 | g | 1.0723 | 105.32% |
| Lean Mass | 9 | 5-6 | 0.0216 | 14.6667 | g | 0.4633 | 94.63% |
| Lean Mass | 20 | 4 | 0.792 | 20.5250 | g | 2.0105 | 98.43% |
| Body Mass | 6 | 10 | 0.710 | 16.6125 | g | 1.1281 | 100.99% |
| Body Mass | 9 | 5-6 | 0.0277 | 16.6333 | g | 0.4803 | 94.72% |
| Body Mass | 20 | 4 | 0.0917 | 25.2250 | g | 2.6763 | 87.74% |
| Fat Mass | 6 | 10 | 0.000477 | 2.0375 | g | 0.1061 | 78.37% |
| Fat Mass | 9 | 5-6 | 0.477 | 1.9833 | g | 0.1472 | 95.35% |
| Fat Mass | 20 | 4 | 0.00714 | 4.7500 | g | 0.7594 | 60.32% |
| % Fat | 6 | 10 | 0.000195 | 12.2875% | | 0.8202 | 78.07% |
| % Fat | 9 | 5-6 | 0.927 | 11.9% | | 0.7305 | 100.48% |
| % Fat | 20 | 4 | 0.00221 | 18.7% | | 1.2754 | 68.50% |
| Bone Density | 6 | 10 | 0.3432 | 0.0425 | g/cm² | 0.0012 | 98.72% |
| Bone Density | 9 | 5-6 | 0.0980 | 0.0441 | g/cm² | 0.0014 | 96.90% |
| Bone Density | 20 | 4 | 0.270 | 0.0583 | g/cm² | 0.0033 | 103.92% |
| Bone Calcium | 6 | 10 | 0.3729 | 0.2988 | g | 0.0162 | 102.17% |
| Bone Calcium | 9 | 5-6 | 0.216 | 0.3305 | g | 0.0198 | 94.97% |
| Bone Calcium | 20 | 4 | 0.0254 | 0.5415 | g | 0.0611 | 121.34% |
| Bone Area | 6 | 10 | 0.0733 | 6.9813 | cm² | 0.2832 | 102.92% |
| Bone Area | 9 | 5-6 | 0.37225 | 7.4983 | cm² | 0.3092 | 97.51% |
| Bone Area | 20 | 4 | 0.00609 | 9.2600 | cm² | 0.5062 | 116.30% |
| Femur Density | 9 | 5-6 | 0.100 | 0.0593 | g/cm² | 0.0040 | 95.13% |
| Femur Density | 20 | 4 | 0.0351 | 0.1039 | g/cm² | 0.0108 | 111.38% |
| Femur Calcium | 9 | 5-6 | 0.0306 | 0.0192 | g | 0.0010 | 94.36% |
| Femur Calcium | 20 | 4 | 0.00845 | 0.0345 | g | 0.0046 | 120.00% |
| Femur Area | 9 | 5-6 | 0.846 | 0.3283 | cm² | 0.0175 | 100.41% |
| Femur Area | 20 | 4 | 0.0646 | 0.3325 | cm² | 0.0282 | 108.13% |
| Tibia Density | 9 | 5-6 | 0.00723 | 0.0477 | g/cm² | 0.0018 | 95.31% |
| Tibia Density | 20 | 4 | 0.00656 | 0.0719 | g/cm² | 0.0059 | 112.39% |
| Tibia Calcium | 9 | 5-6 | 0.0258 | 0.0195 | g | 0.0011 | 94.20% |
| Tibia Calcium | 20 | 4 | 0.00118 | 0.0321 | g | 0.0034 | 119.53% |
| Tibia Area | 9 | 5-6 | 0.743 | 0.4042 | cm² | 0.0202 | 99.30% |
| Tibia Area | 20 | 4 | 0.0931 | 0.4450 | cm² | 0.0169 | 105.01% |

Observation of body mass in an additional strain of mice confirmed that the difference in body fat is TLR4-dependent. Mice in which the TLR4 mutation of C3H/HeJ was crossed onto a Balb/c mouse background (C.C3H-TLR4-lpsd strain available from Jackson Labs) also had significantly less body fat, and similar lean body mass at 6 weeks of age (see Table 4).

TABLE 4
TLR4 Mutant Congenic on Balb/c background (C.C3H-TLR4-lpsd)

| Measurement | Age (wks) | N | P value | Average | | St. Dev | % of WT |
|---|---|---|---|---|---|---|---|
| Lean Mass | 6 | 10 | 0.089 | 13.79 | g | 0.8749 | 105.19% |
| Body Mass | 6 | 10 | 0.809 | 15.95 | g | 0.9880 | 100.69% |
| Fat Mass | 6 | 10 | 0.000399 | 2.17 | g | 0.2406 | 79.49% |
| % Fat | 6 | 10 | 8 × 10⁻⁶ | 13.5% | | 1.1695 | 78.48% |
| Bone Density | 6 | 10 | 0.761 | 0.045 | g/cm² | 0.0022 | 99.28% |
| Bone Calcium | 6 | 10 | 0.165 | 0.2723 | g | 0.0304 | 106.70% |
| Bone Area | 6 | 10 | 0.0113 | 6.544 | cm² | 0.4337 | 107.35% |

Each of the strains of mice was routinely tested for numerous infections as infections can lead to loss of muscle and total body weight. No infections were observed and the mice continued to grow throughout the analysis. This was confirmed by comparing age and sex matched mice in the mouse facility with mice in the super sanitary Barrier facility. The mice in the barrier facility showed the same TLR4 dependent body fat differences, in fact more so than those in the regular animal facility by the age of 12 weeks. All mice appeared healthy and reproduced effectively, with similar numbers of offspring to wild-type control mice. Together these data indicate that TLR4 is a master regulator of fat body mass, and that loss of TLR4 signaling may result in inhibition of gains in fat or even loss of body fat.

### Example 16

### TLR4 Activity and Metabolic Syndrome

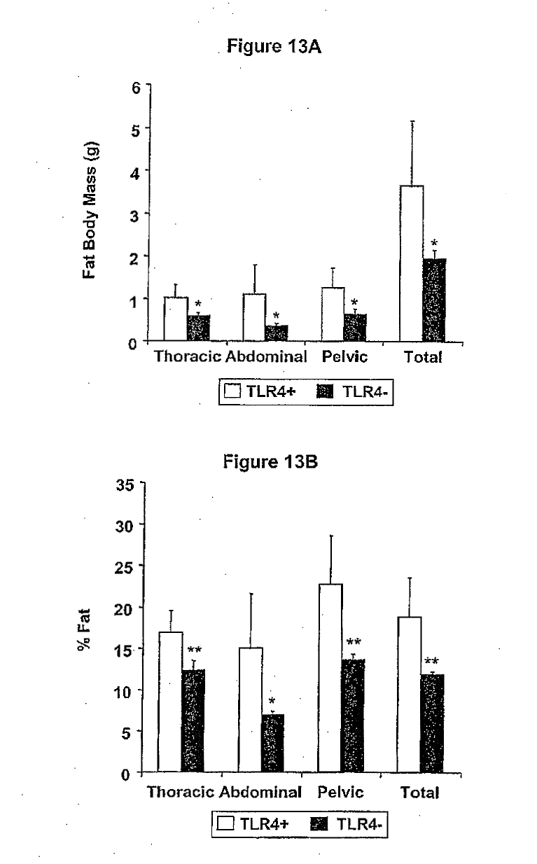

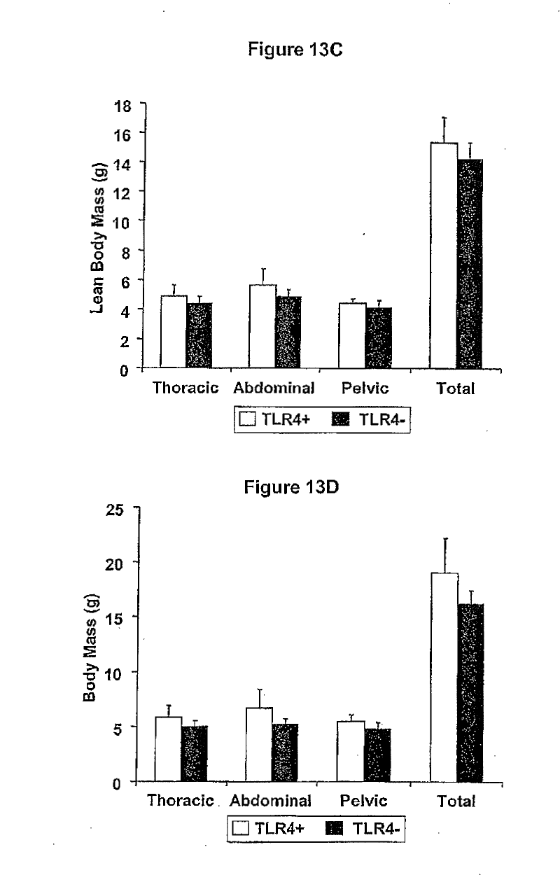

Metabolic syndrome is associated with general obesity, but is more significantly associated with central or abdominal obesity. In addition, central adiposity is a greater risk factor for type two diabetes than general obesity. The distribution of adiposity in wild type and TLR mutant mice was evaluated. Two groups of 5 female mice at 8 weeks of age (C3H/HeJ and C3H/HeSnJ) were analyzed by DEXA, and the data were separated based on body segment. All body segments showed significantly less body fat and percent body fat in the TLR4 mutant mice than in the wild type mice, with the abdominal segment showing the greatest difference in adiposity (FIGS. 13A and 13B and Table 5). No difference was observed in lean body mass or total body mass in any segment or in the whole animals (FIGS. 13C and 13D and Table 5). Thus, the central adiposity of the TLR4 mutant mice was more reduced than adiposity in any other area. These data suggest that TLR4 may regulate the processes that cause central adiposity and metabolic syndrome. Therefore, TLR4 inhibition or modification may be useful to treat metabolic syndrome and type two diabetes regardless of effect on general levels of adiposity.

TABLE 5

| | Fat Body Mass (g) | Percent Body Fat | Lean Body Mass (g) | Body Mass (g) |
|---|---|---|---|---|
| Thoracic | −41.2% (P = 0.02) | −26.7% (P = 0.006) | −9.4% (P = 0.3) | −13.7% (P = 0.2) |
| Abdominal | −67.3% (P = 0.04) | −53.3% (P = 0.03) | −13.2% (P = 0.2) | −21.5% (P = 0.1) |
| Pelvic | −49.2% (P = 0.02) | −39.7% (P = 0.01) | −5.9% (P = 0.3) | −12.7% (P = 0.1) |
| Total | −46.4% (P = 0.04) | −36.6% (P = 0.01) | −6.9% (P = 0.3) | −14.6% (P = 0.1) |

Values represent the percent difference between TLR4 mutant mice and wild type mice.

### Example 17

### Bone Density of Mice Lacking TLR4

Bone density, bone area, and bone calcium were examined in the three strains of TLR4 mutant mice described above and compared to that of age and sex matched control mice having a functional TLR4. Bone density, bone calcium content and bone area were measured by dual x-ray absorptometry using a PIXIMUS small animal densitometer (LUNAR, Madison, Wis.). Mice were either euthanized or anesthetized by IP injection according to IUCAC approved procedures. All measurements were taken in live anesthetized mice or in euthanized mice. Data analysis was done with PIXIMUS software. All bone measurements excluded the skull, as recommended by LUNAR. Tibia and femur measurements were obtained by measuring bone parameters within a region of interest surrounding the right or left tibia or femur of each mouse. The same skeletal landmarks were used to select the region of interest in both controls and mutant mice. As indicated in Tables 2-3, mice with mutations in TLR4 had significantly increased bone mineral density, bone mineral content, and bone area, as measured by dual x-ray absorptometry. TLR4 mutations lead to higher bone mineral density and higher bone mineral content despite similar total body weights. Given the strong positive correlation in mammals of body fat and bone mineral density, it was unexpected that these mutant mice would have higher bone density and lower percent body fat. Mutant mice also had bones with larger area. These differences were not present in all of the mice.

### Example 18

### Activity of Mice Lacking TLR4

Activity of the mice lacking TLR4 and control mice was assessed using an activity box, CCDIGI/DIGIPROI System version 1.30 (Accuscan Instruments, Inc.). Mice with the TLR4 mutation (17 wk old female C3H/HeJ, N=10) were less active than TLR4 wild-type mice (17 wk-old female C3H/HeSnJ, N=10). The data were statistically significant by a number of different readouts (p≦0.001; see Table 6). Surprisingly, the distance traveled per hour (the best measure of kinetic energy expenditure) was about 50% less in the TLR4 mutants. These data show that the TLR4 mutant mice were not only leaner (less body fat), but were also less physically active than the TLR4 wild-type mice. Thus, the mutation in TLR4 does not have its effects on body fat and bone due to an increase in their physical activity.

TABLE 6
TLR4 Mutant (C3H/HeJ)

| | P value | Measurement | | St. Dev. | % of WT |
|---|---|---|---|---|---|
| Distance | 0.0011 | 493 | cm/hr | 132 | 44% |
| traveled | | | | | |
| Sterotypy | 0.00018 | 1,222 | cts/hr | 177 | 59% |
| Counts | | | | | |
| Movement | 0.0024 | 59.60 | sec/hr | 14.66 | 52% |
| Time | | | | | |
| # of | 0.021 | 95.98 | /hr | 22.94 | 63% |
| Movements | | | | | |

### Example 19

### CD14 Acts with TLR4 in Regulating Body Fat and Bone Density

To confirm the results showing decreases in body fat and increases in bone density and mineral content with a loss-of-function of TLR4, CD14 knockout mice (B6.129S-Cd14^{tm1Frm}) were analyzed and compared with the control strain, C57B1/6J, using dual x-ray absorptometry. CD14 knockout mice and C57B1/6J control mice were purchased from Jackson Labs. The CD14 knockout mice have been backcrossed 20 times onto the C57B1/6J strain. The TLR4 mutant phenotype of high bone mineral density and low % body fat also was present in CD14 knockout mice (see Table 7). This indicates that the TLR/CD14 receptor complex regulates body fat and bone density. The body fat and % fat differences were significant at 6 weeks of age but were not significant at 12 weeks of age.

TABLE 7

| | | | | | | |
|---|---|---|---|---|---|---|
| | Age | | | Average | St. | |
| Measurement | (wks) | N | P value | Value | Dev | % of WT |

CD14 Knock-out (B6.129S-Cd14^{tm1Frm})

| | | | | | | | |
|---|---|---|---|---|---|---|---|
| Lean Mass | 6 | 4-5 | 0.00957 | 12.98 | g | 0.46 | 106.3% |
| Lean Mass | 12 | 10 | 0.0000152 | 15.49 | g | 0.66 | 112.9% |
| Body Mass | 16 | 4-5 | 0.724 | 14.98 | g | 0.65 | 100.8% |
| Body Mass | 12 | 10 | 0.000231 | 18.46 | g | 0.90 | 110.8% |
| Fat Mass | 6 | 4-5 | 0.00180 | 1.96 | g | 0.18 | 73.5% |
| Fat Mass | 12 | 10 | 0.785 | 2.99 | g | 0.31 | 101.7% |

| | | | | | | |
|---|---|---|---|---|---|---|
| % Fat | 6 | 4-5 | 0.000386 | 13.14% | 1.01 | 73.5% |
| % Fat | 12 | 10 | 0.109 | 16.16% | 1.09 | 91.9% |

CD14 Knock-out (B6.129S-Cd14^{tm1Frm})

| | | | | | | | |
|---|---|---|---|---|---|---|---|
| Total Bone Density | 6 | 4-5 | 0.0062 | 0.0424 | g/cm² | 0.0015 | 111.68% |
| Total Bone Density | 12 | 10 | 3.65 × 10⁻⁸ | 0.0488 | g/cm² | 0.0013 | 110.44% |
| Total Bone Calcium | 6 | 4-5 | 0.0010 | 0.2820 | g | 0.0124 | 126.46% |
| Total Bone Calcium | 12 | 10 | 3.35 × 10⁻⁷ | 0.3473 | g | 0.0013 | 121.73% |
| Total Bone Area | 6 | 4-5 | 0.00083 | 6.6520 | cm² | 0.1588 | 113.32% |
| Total Bone Area | 12 | 10 | 0.00019 | 7.1170 | cm² | 0.2915 | 110.20% |

Bone density, bone calcium content, bone area, moment of inertia and moment of resistance of the mid-shaft (mid-diaphysis) of the right tibia (9.2 mm from the proximal end of each tibia) of the CD14 knockout mice were measured by peripheral quantitative computed tomography (pQCT) using a XCT Research SA+pQCT scanner (STRATEC Medizinetechnik GmbH, Durlacher, Germany). All mice were 13 weeks and 5 days old, and were female. Mice were anesthetized by IP injection. Data analysis was done with STRATEC software version 5.40. The same skeletal landmarks were used in all measurements. Results are presented in Table 8.

TABLE 8
CD14 Knock-out (B6.129S-Cd14^{tm1Frm})

| | Average | | | | % of | |
|---|---|---|---|---|---|---|
| Parameter | Value | | St. Dev. | | WT | P Value |
| Total Bone Content | 0.916 | mg | 0.0368 | 9/9 | 109.87 | 0.00036 |
| Cortical and Subcortical Bone | 0.857 | mg | 0.0346 | 9/9 | 108.44 | 0.0015 |
| Content | | | | | | |
| Trabecular Bone Content | 0.056 | mg | 0.0073 | 9/9 | 125.00 | 0.0019 |
| Cortical Bone Content | 0.690 | mg | 0.0300 | 9/9 | 109.52 | 0.0031 |
| Total Bone Density | 701.867 | mg/mm² | 16.9031 | 9/9 | 97.21 | 0.031 |
| Cortical and Subcortical Bone | 853.556 | mg/mm² | 13.7198 | 9/9 | 100.50 | 0.69 |
| Density | | | | | | |
| Trabecular Bone Density | 190.600 | mg/mm² | 8.5481 | 9/9 | 97.87 | 0.55 |
| Cortical Bone Density | 1091.800 | mg/mm² | 15.8536 | 9/9 | 98.89 | 0.17 |
| Total Bone Area | 1.304 | mm² | 0.0702 | 9/9 | 112.78 | 0.00012 |
| Cortical and Subcortical Bone | 1.007 | mm² | 0.0381 | 9/9 | 108.11 | 0.0014 |
| Area | | | | | | |
| Trabecular Bone Area | 0.299 | mm² | 0.0344 | 9/9 | 131.22 | 4.4 × 10⁵ |
| Cortical Bone Area | 0.631 | mm² | 0.0247 | 9/9 | 110.51 | 0.0012 |
| Mean Cortical Thickness | 0.182 | mm | 0.0040 | 9/9 | 103.41 | 0.14 |
| Periosteal Circumference | 4.048 | mm | 0.1079 | 9/9 | 106.30 | 9.0 × 10⁵ |
| Endosteal Circumference | 2.091 | mm | 0.1063 | 9/9 | 107.46 | 0.00057 |
| Polar Moment of Inertia of | 0.284 | mm⁴ | 0.0288 | 9/9 | 126.73 | 8.1 × 10⁵ |
| Total Bone | | | | | | |
| Polar Moment of Inertia of | 0.143 | mm⁴ | 0.0150 | 9/9 | 132.99 | 4.4 × 10⁵ |
| Cortical Bone | | | | | | |
| Polar Moment of Inertia of | 0.129 | mm⁴ | 0.0154 | 9/9 | 130.34 | 0.00026 |
| Weighted Cortical Bone | | | | | | |
| X* Axial Moment of Inertia of | 0.143 | mm⁴ | 0.0158 | 9/9 | 132.99 | 3.0 × 10⁵ |
| Total Bone | | | | | | |
| X Axial Moment of Inertia of | 0.071 | mm⁴ | 0.0105 | 9/9 | 142.22 | 0.00013 |
| Cortical Bone | | | | | | |
| X Axial Moment of Inertia of | 0.067 | mm⁴ | 0.0087 | 9/9 | 139.53 | 9.0 × 10⁵ |
| Weighted Cortical Bone | | | | | | |
| Y Axial Moment of Inertia of | 0.142 | mm⁴ | 0.0274 | 9/9 | 119.63 | 0.0039 |
| Total Bone | | | | | | |
| Y Axial Moment of Inertia of | 0.071 | mm⁴ | 0.0071 | 9/9 | 123.08 | 0.0030 |
| Cortical Bone | | | | | | |
| Y Axial Moment of Inertia of | 0.63 | mm⁴ | 0.0088 | 9/9 | 123.91 | 0.0031 |
| Weighted Cortical Bone | | | | | | |
| Polar Moment of Resistance of | 0.33 | mm³ | 0.032 | 9/9 | 123.05 | 0.00041 |
| Total Bone | | | | | | |
| Polar Moment of Resistance of | 0.22 | mm³ | 0.016 | 9/9 | 122.01 | 8.7 × 10⁵ |
| Cortical Bone | | | | | | |
| Polar Moment of Resistance of | 0.19 | mm³ | 0.014 | 9/9 | 121.83 | 8.1 × 10⁵ |
| Weighted Cortical Bone | | | | | | |
| X Axial Moment of Resistance | 0.21 | mm³ | 0.027 | 9/9 | 123.33 | 0.0012 |
| of Total Bone | | | | | | |
| X Axial Moment of Resistance | 0.12 | mm³ | 0.007 | 9/9 | 122.09 | 4.0 × 10⁵ |
| of Cortical Bone | | | | | | |
| X Axial Moment of Resistance | 0.11 | mm³ | 0.009 | 9/9 | 123.38 | 0.00019 |
| of Weighted Cortical Bone | | | | | | |
| Y Axial Moment of Resistance | 0.20 | mm³ | 0.020 | 9/9 | 116.99 | 0.0050 |
| of Total Bone | | | | | | |
| Y Axial Moment of Resistance | 0.11 | mm³ | 0.008 | 9/9 | 116.09 | 0.0088 |
| of Cortical Bone | | | | | | |
| Y Axial Moment of Resistance | 0.10 | mm³ | 0.007 | 9/9 | 116.88 | 0.0050 |
| of Weighted Cortical Bone | | | | | | |

*X axis is Anterior-Posterior, Y Axis is Lateral

Polar moment of resistance (by pQCT) and density (by dual X ray absorptometry) are well correlated with bone failure strength. Both of these parameters predict significantly stronger bones in CD14 knockout animals. Taken as a whole, the dual X ray absorptometry and pQCT data indicate that the CD14 knockout mice have stronger bones then wild-type, but differ in their measurements of bone density. Bone density measurements by pQCT show no difference, while bone density measurements by dual X ray absorptometry show significant differences. Both dual X ray absorptometry and pQCT show significantly more total bone content in CD14 knockout mice compared to wild-type controls.

### Example 20

### TLR4/CD14 Regulates Bone Stiffness and Resistance to Fracture

To confirm that the increased bone density and mineral content in the mutant mice correlates with actual increased bone strength, tibias from CD14 knockout mice were compared to control mice. Stiffness, elastic modulus and maximum force sustainable before fracture of tibias were measured by three-point biomechanical testing as follows. Mouse tibias were freshly dissected and mechanically tested in a 3-point bending configuration to determine their flexural properties. Testing was performed using a Dynamic Mechanical Analyzer (DMA 2980, New Castle, Del.). An increasing load was applied, at a rate of 0.1 N per second, to the anterior aspect of each tibia diaphysis until failure. Specimens were immersed in saline before and during testing. Using the Euler-Bernoulli beam formulation, the slope of the force-deflection curve was used to calculate the bone's bending rigidity (EI) (eqn. 1).

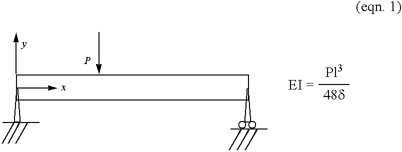

Where P=applied load, δ=beam deflection at mid-span, l=beam distance between outer supports, E=Young's modulus, I=area moment of inertia.

To determine material properties, each tibia was imaged by cross-section by pQCT using a XCT Research SA+pQCT scanner (STRATEC Medizinetechnik GmbH, Durlacher, Germany). This cross-sectional data was used to calculate the moment of inertia (I) near the tibia mid-span using STRATEC software version 5.40. The moment of inertia was used in Equation 1 to determine the Young's modulus (E) in bending.

Tibias from mutant mice have increased stiffness and can bear a higher maximum load before fracture (see Table 9). This suggests that blockade of TLR4/CD14 can result in changes in bone that reduce the incidence of fracture, as commonly occur in osteoporosis and other bone disorders. The elastic modulus of mutant bones was decreased, but this difference was not significant according to these tests. These data suggest that bones from mice with mutations in the TLR4/CD14 receptor complex have normal molecular architecture of their bones. This is as opposed to what is seen in osteopetrosis, where bones are denser, but are also more brittle. These data suggest that drug therapy targeted at inhibiting TLR4/CD14 for extended periods of time will result in increased bone density and strength without resulting in poor bone architecture, or brittleness.

TABLE 9
CD14 Knockout (B6.129S-Cd14^{tm1Frm})

| Measurement | Age (wks) | N | P value | Average | | St. Dev | % of WT |
|---|---|---|---|---|---|---|---|
| Stiffness | 4 | 8 | 0.00652 | 53.1 | N/mm | 8.17 | 114.15% |
| Elastic Modulus | 4 | 8 | 0.126 | 11.4 | GPa | 1.68 | 92.20% |
| Maximum Force | 4 | 8 | 6.33 × 10⁻⁷ | 11.22 | N | 0.863 | 117.21% |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a compound that increases bone density, said method comprising:
(a) contacting a cell with a test compound and monitoring the activity of a Toll-Like Receptor (TLR) in said cell in response to an agonist;
(b) administering said compound to a non-human subject if activity of said TLR in said cell is reduced relative to the level of activity of said TLR in the absence of said compound; and
(c) identifying said compound as useful for increasing bone density if bone density in said non-human subject is increased relative to bone density in a corresponding subject to which said test compound was not administered.

2. The method of claim 1, wherein said TLR is TLR2, TLR4, or TLR9.

3. The method of claim 1, wherein said TLR is TLR4.

4. The method of claim 1, wherein said test compound is a glycosaminoglycan, a glycoprotein, a polysaccharide, a polypeptide, or a nucleic acid.

5. The method of claim 4, wherein said glycoprotein comprises hyaluronic acid.

6. The method of claim 5, wherein said test compound is a hyaluronic acid-protein conjugate.

7. The method of claim 4, wherein said glycoprotein comprises heparan sulfate (HS).

8. The method of claim 7, wherein said test compound is a HS-protein conjugate.

9. The method of claim 4, wherein said glycoprotein comprises chondroitin sulfate.

10. The method of claim 4, wherein said polypeptide is an anti-CD14 antibody.

11. The method of claim 4, wherein said nucleic acid is polymerized.

12. The method of claim 1, wherein said test compound is a protease inhibitor.

13. The method of claim 12, wherein said protease inhibitor is an elastase inhibitor.

14. The method of claim 1, wherein said test compound is a heparanase.

15. The method of claim 1, wherein said test compound modulates the sulfation of HS.

16. The method of claim 1, wherein said test compound is a lipid A analogue.

17. The method of claim 1, wherein monitoring TLR activity comprises measuring expression of a cytokine or a chemokine.

18. The method of claim 1, wherein said non-human subject is a rodent.

19. A method of identifying a compound that decreases fat mass, said method comprising:
(a) contacting a cell with a test compound and monitoring the activity of a TLR in said cell in response to an agonist;
(b) administering said compound to a non-human subject if activity of said TLR in said cell is reduced relative to the level of activity of said TLR in the absence of said compound, and
(c) identifying said compound as useful for decreasing fat mass if the fat mass in said non-human subject is decreased relative to the fat mass in a corresponding subject to which said test compound was not administered.

20. A method of identifying a compound for treatment of osteoporosis or obesity, said method comprising
(a) administering a test compound to a non-human subject;
(b) monitoring activity of a TLR in response to an agonist in said non-human subject; and
(c) identifying said test compound as useful for treatment of osteoporosis or obesity if activity of said TLR is decreased in said non-human subject relative to that of a corresponding non-human subject to which said test compound was not administered.

21-59. (canceled)


SEQUENCE LISTING (6 sequences)

SEQ ID NO: 1 - DNA, 30 bp, Artificial Sequence
  Primer
cgcggatccaggatgatgcctccctggctc

SEQ ID NO: 2 - DNA, 30 bp, Artificial Sequence
  Primer
ggcggtacctcaggtccaagttgccgtttc

SEQ ID NO: 3 - DNA, 20 bp, Artificial Sequence
  Primer
ccggaattcatcatgttgcc

SEQ ID NO: 4 - DNA, 24 bp, Artificial Sequence
  Primer
ccggaattcctaattgacatcacg

SEQ ID NO: 5 - DNA, 30 bp, Artificial Sequence
  Primer
ccggaattcaccatggagcgtgtgcttggc

SEQ ID NO: 6 - DNA, 33 bp, Artificial Sequence
  Primer
ccggaattcttaaacaaagaggcgatctcctag